(19) United States
(12) Patent Application Publication
(10) Pub. No.: US 2005/0148100 A1
(43) Pub. Date: Jul. 7, 2005

(54) Methods and devices for using Raman-active probe constructs to assay biological samples

(75) Inventors: Xing Su, Cupertino, CA (US); Andrew A. Berlin, San Jose, CA (US); Tae-Woong Koo, South San Francisco, CA (US); Selena Chan, San Jose, CA (US); Lei Sun, Santa Clara, CA (US)

Correspondence Address: DLA PIPER RUDNICK GRAY CARY US, LLP, 4365 EXECUTIVE DRIVE, SUITE 1100, SAN DIEGO, CA, 92121-2133, US

(73) Assignee: Intel Corporation, Santa Clara, CA (US)

(21) Appl. No.: 10/750,301

(22) Filed: Dec. 30, 2003

(51) Int. Cl. G01N 33/53; G01N 33/543

(52) U.S. Cl. 436/523

(57) ABSTRACT

Various methods of using Raman-active or SERS-active probe constructs to detect analytes in biological samples, such as the protein-containing analytes in a body fluid are provided. The probe moieties in the Raman-active constructs are selected to bind to and identify specific known analytes in the biological sample or the probe moieties are designed to chemically interact with functional groups commonly found in certain amino acids so that the invention methods provide information about the amino acid composition of protein-containing analytes or fragments in the samples. In some cases, the Raman-active or SERS-active probe constructs, when used in the invention methods, can identify particular protein-containing analytes or types of such analytes so that a protein profile of a patient sample can be made. When compared to a data base of Raman or SERS spectra of normal samples, a disease state of a patient can be identified using the methods disclosed.

### BACKGROUND OF THE INVENTION

1. Field of the Invention

The present invention relates generally to methods and devices useful to identify the presence of an analyte in a sample and, more specifically to methods and devices for use of Raman-active probe constructs to assay biological samples.

2. Background Information

The remarkable success of genome level DNA sequencing has placed us at a threshold of knowledge that was unimaginable 25 years ago. To enable this watershed of data to be transformed into knowledge that will be of use in diagnosing, staging, understanding, and treating human diseases will require that we not only know the sequences of the estimated >30,000 human proteins but also that we identify key changes in protein expression which portend the impending onset of disease, accurately classify at the molecular level the disease subtype, and that we understand the functions, interactions, and how to modulate the activities of proteins which are intimately involved in disease processes. One of the most fundamental approaches to understanding protein function is to correlate expression level changes as a function of growth conditions, cell cycle stage, disease state, external stimuli, level of expression of other proteins, or other variable. Although DNA microarray analysis offers a massively parallel approach to genome-wide mRNA expression analysis, there often is not a direct relationship between the in vivo concentration of an mRNA and its encoded protein. Differential rates of translation of mRNAs into protein and differential rates of protein degradation in vivo are two factors that confound the extrapolation of mRNA to protein expression profiles.

Additionally, such microarray analysis is unable to detect, identify or quantify post-translational protein modifications—which often play a key role in modulating protein function. Protein expression analysis offers a potentially large advantage in that it measures the level of the biological effecter protein molecule, not just that of its message. Currently, no protein profiling technology is available that can approach the ability of microarray analysis to simultaneously profile the relative level of mRNA expression of 25,000 or more genes.

Thus, ever increasing attention is being paid to detection and analysis of low concentrations of analytes in various biologic and organic environments. Qualitative analysis of such analytes is generally limited to the higher concentration levels, whereas quantitative analysis usually requires labeling with a radioisotope or fluorescent reagent. Such procedures are generally time consuming and inconvenient.

Solid-state sensors and particularly biosensors have received considerable attention lately due to their increasing utility in chemical, biological, and pharmaceutical research as well as disease diagnostics. In general, biosensors consist of two components: a highly specific recognition element and a transducing structure that converts the molecular recognition event into a quantifiable signal. Biosensors have been developed to detect a variety of biomolecular complexes including oligonucleotide pairs, antibody-antigen, hormone-receptor, enzyme-substrate and lectin-glycoprotein interactions. Signal transductions are generally accomplished with electrochemical, field-effect transistor, optical absorption, fluorescence or interferometric devices.

It is known that the intensity of the visible reflectivity changes of a porous silicon film can be utilized in a simple biological sensor for possible detection, identification and quantification of small molecules. While such a biological sensor is certainly useful, detection of a reflectivity shift is complicated by the presence of a broad peak rather than one or more sharply defined luminescent peaks.

Raman spectroscopy or surface plasmon resonance has also been used seeking to achieve the goal of sensitive and accurate detection or identification of individual molecules from biological samples. When light passes through a medium of interest, a certain amount of the light becomes diverted from its original direction in a phenomenon known as scattering. Some of the scattered light also differs in frequency from the original excitatory light, due to the absorption of light and excitation of electrons to a higher energy state, followed by light emission at a different wavelength. The difference of the energy of the absorbed light and the energy of the emitted light matches the vibrational energy of the medium This phenomenon is known as Raman scattering, and the method to characterize and analyze the medium or molecule of interest with the Raman scattered light is called Raman spectroscopy. The wavelengths of the Raman emission spectrum are characteristic of the chemical composition and structure of the Raman scattering molecules in a sample, while the intensity of Raman scattered light is dependent on the concentration of molecules in the sample.

A Raman spectrum, similar to an infrared spectrum, consists of a wavelength distribution of bands corresponding to molecular vibrations specific to the sample being analyzed (the analyte). In the practice of Raman spectroscopy, the beam from a light source, generally a laser, is focused upon the sample to thereby generate inelastically scattered radiation, which is optically collected and directed into a wavelength-dispersive spectrometer in which a detector converts the energy of impinging photons to electrical signal intensity.

Historically, the very low conversion of incident radiation to inelastic scattered radiation limited Raman spectroscopy to applications that were difficult to perform by infrared spectroscopy, such as the analysis of aqueous solutions. It was discovered however, that when a molecule in close proximity to a roughened silver electrode is subjected to a Raman excitation source the intensity of the signal generated is increased by as much as six orders of magnitude.

Although the mechanism responsible for this large increase in scattering efficiency is currently the subject of considerable research, it is generally accepted that the phenomenon occurs if the following three conditions are satisfied: (1) that the free-electron absorption of the metal can be excited by light of wavelength between 250 and 2500 nanometers (nm), preferably in the form of laser beams; (2) that the metal employed is of the appropriate size (normally 5 to 1000 nm diameter particles, or a surface of equivalent morphology), and has optical properties necessary for generating a surface plasmon field; and (3) that the analyte molecule has effectively matching optical properties (absorption) for coupling to the plasmon field.

In particular, nanoparticles of gold, silver, copper and certain other metals can function to enhance the localized effects of electromagnetic radiation. Molecules located in the vicinity of such particles exhibit a much greater sensitivity for Raman spectroscopic analysis. SERS is the technique to utilize this surface enhanced Raman scattering effect to characterize and analyze biological molecules of interest.

Sodium chloride and lithium chloride have been identified as chemicals that enhance the SERS signal when applied to a metal nanoparticle or metal coated surface before or after the molecule of interest has been introduced. However, the technique of using these chemical enhancers has not proved sensitive enough to reliably detect low concentrations of analyte molecules, such as single nucleotides or proteins, and as a result SERS has not been suitable for analyzing the protein content of a complex biological sample, such as blood plasma.

Thus a need exists in the art for a method of analyte detection that provides more information regarding the characteristics of the bound analyte and for reliably detecting and/or identifying individual analytes using a Raman spectroscopic analytical technique. In addition, there is also a need in the art for quick and simple means of qualitatively and quantitatively detecting biomolecules at low concentration levels.

### BRIEF DESCRIPTION OF THE DRAWINGS

FIG. 1 is a schematic drawing showing a prior art polymer gel (top) with an invention gel impregnated with silver nanoparticles (bottom).

FIG. 2 is a schematic flow chart illustrating preparation and separation of invention optical barcodes with attached antibody probes. When contacted with a variety of target molecules, the antibodies in the optical barcode constructs specifically bind to different analytes. The probed complexes so formed are shown as subjected to 2-dimensional separation in preparation for detection of optical signals, such as Raman signals, generated by illumination of the separated complexes.

FIG. 3 is a schematic diagram illustrating how the four function roles of the invention active molecular Raman codes interrelate with the four structural domains of the probe construct.

FIG. 4 is a graph of three Raman SERS spectra that illustrate the effect on the Raman signal from invention active molecular Raman codes obtained by changing number, and position of Raman-active tags on single strand oligonucleotide backbones of equal lengths (21 residues). RR1 is the Raman signal of a construct having two Raman tags, ROX and FAM, positioned at opposite ends of the oligonucleotide backbone, but lacking an amino group enhancer. AT3, AT11 and AT19 are Raman spectra of three constructs that share a common backbone and a 5′ amino group enhancer, but the same Raman tag, TAMRA, is positioned at three different locations along the backbone.

FIG. 5 is graph of two Raman SERS spectra that illustrate the effect of enhancers in invention active molecular Raman codes. The constructs that produced spectra PGPT and NPGPT both have a linear single strand poly(dT) backbone with a poly(dG) Raman tag of 10 residues. PGPT lacks an enhancer group, while NPGPT has an enhancer moiety (AmC6) attached to the poly(dG) Raman tag to enhance the Raman signal provided by the poly(dG) Raman tag.

FIGS. 6A-B are graphs of Raman SERS spectra that illustrate crossover effects of functional/structural domains in invention active molecular Raman codes. The SPTA spectrum shown in FIG. 6A is generated by a molecular construct having a ThiSS active group, a poly(dT) backbone, and a single dA tag at the 5′ end. The spectrum shows that that Raman active moiety is the single dA residue with the molecular backbone provides a slight enhancer function. The ACRGAM spectrum shown in FIG. 6B is generated by a molecular construct having a 5Acrd active group, a poly(dG) backbone, and a single AmC7 as an enhancer group at the 5′ end. The Raman spectrum is produced primarily by the Raman-active poly (dG) backbone with the enhancer amplifying the signal.

FIGS. 7A-D are a series of schematic flow diagrams that illustrate four different embodiments of invention methods to use cascade binding to enhance Raman signal from an immobilized analyte. In each embodiment an active molecular Raman code with primary antibody and DNA backbone immobilizes the analyte on the substrate. FIGS. 7A, 7B, 7C and 7D illustrate, respectively, hybridization of four different types of secondary Raman complex to the Raman-active nucleic acid to enhance the Raman signal: a metal nanoparticle with chemically attached complementary oligonucleotides; a dendrimer formed from complementary oligonucleotides; a double stranded DNA formed by ligation of hybridized oligonucleotides; and subjection of the molecular backbone to terminal transferase reaction using dNTP and Raman tagged oligonucleotides.

FIGS. 8A-I are a series of chemical synthesis diagrams that illustrate use of invention active molecular Raman codes with a DNA Raman active backbone and functional group as the active group to bind specifically to functional groups in amino acid residues in protein-containing molecules.

FIG. 9 is a schematic flow diagram illustrating an invention method to obtain a protein profile of a protein-containing sample. Three active molecular Raman codes specific for amino, sulfhydryl and carboxyl functional groups in amino acids are used.

FIG. 10A illustrates an invention method wherein cascade binding and amplification of a molecular Raman code (as shown in FIGS. 7A-D) is followed by formation of metal nanoparticles in situ to generate SERS signals. FIG. 10B illustrates the intensity of Raman signal produces at three points in the synthesis of the SERS-active construct.

FIG. 11 illustrates a chip with a pool of antibodies distributed at discrete locations. A blowup shows a single discrete location after degenerate cascade binding using a subset of the antibodies in active molecular Raman codes.

FIG. 12 is a schematic flow chart illustrating multiplex analysis of assay results by classification of SERS signatures according to Raman code design. Individual signal points (FIG. 11) can be resolved by performing micro-meter scale SERS scanning and signature analysis, as shown by flow chart in FIG. 12. Comparison of results obtained from control and test samples determines anomalies in the test (patient) sample.

### DETAILED DESCRIPTION OF THE INVENTION

The various embodiments of the invention relate to use of Raman-active or SERS-active probe constructs to detect analytes in biological samples, such as the protein-containing analytes in a body fluid. In certain embodiments, the probe moieties in the Raman-active constructs are selected to bind to and, hence, identify the presence of specific known analytes in the biological sample. In other embodiments, the probe moieties in the Raman-active constructs are designed to chemically interact with functional groups commonly found in certain amino acids so that the invention methods provide information about the amino acid composition of protein-containing analytes or fragments thereof in the samples.

The following detailed description contains numerous specific details in order to provide a more thorough understanding of the disclosed embodiments of the invention. However, it will be apparent to those skilled in the art that the embodiments can be practiced without these specific details. In other instances, devices, methods, procedures, and individual components that are well known in the art have not been described in detail herein.

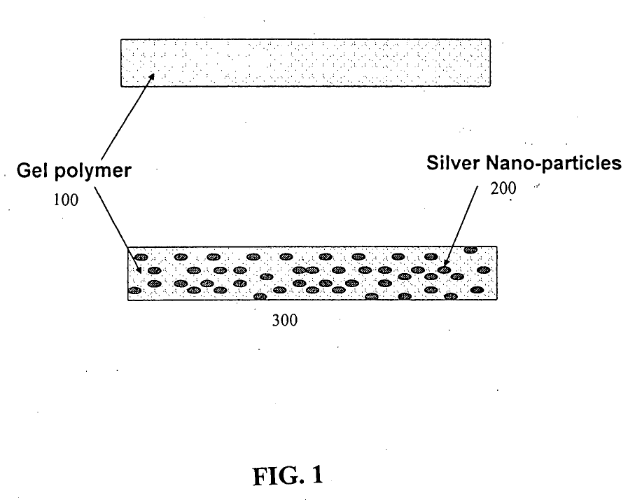

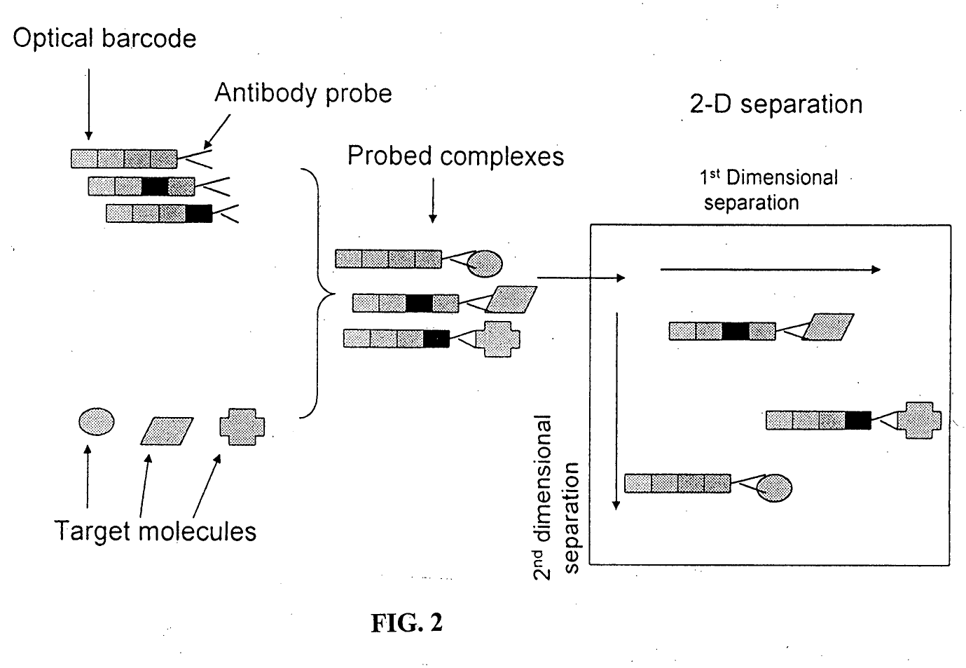

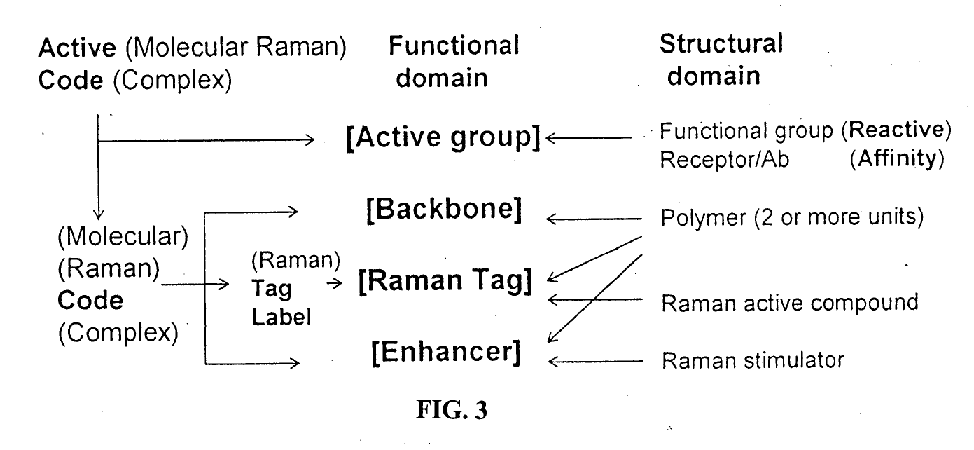

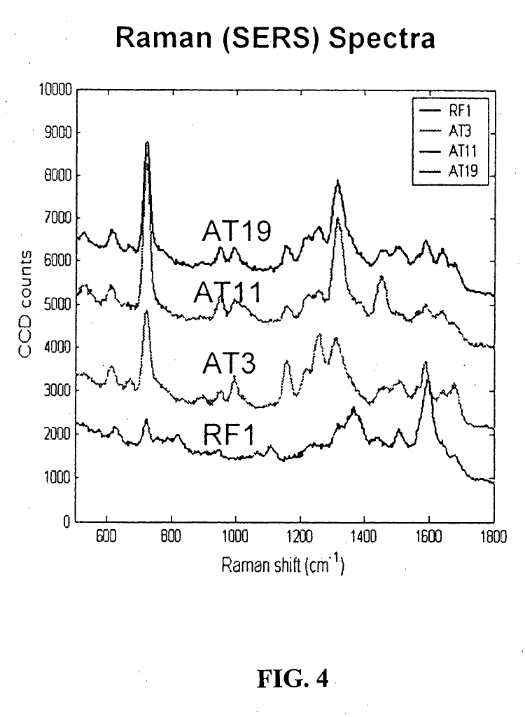

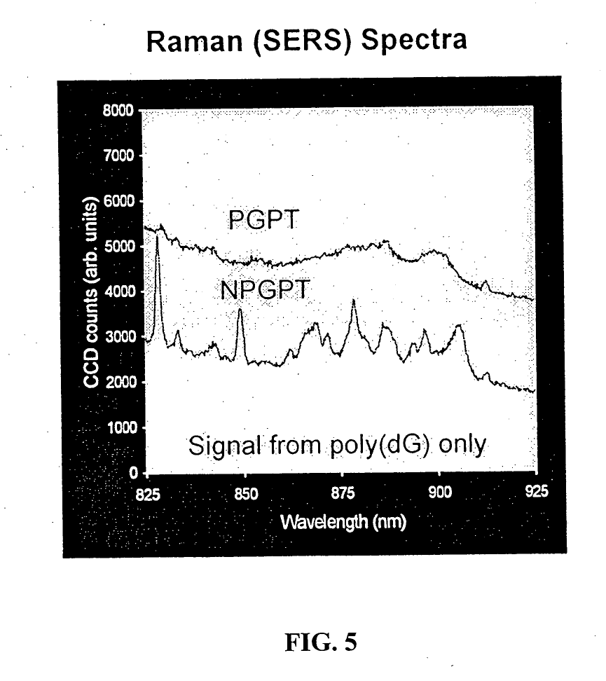

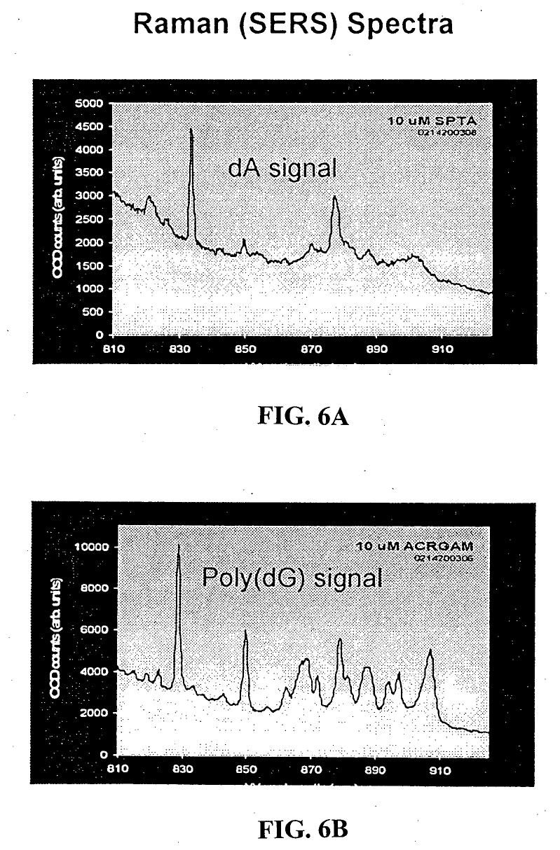

One embodiment of the invention, illustrated in FIG. 1, provides a solid gel matrix 300 comprising a solid gel 100 and one or more SERS-enhancing nanoparticles with an attached probe for binding specifically to an analyte 200. A plurality of the nanoparticles to provide a plurality of unique optical signatures can also be incorporated into the gel matrix. The SERS-enhancing nanoparticles comprise one or more Raman-active tags, as described herein, and a probe that binds specifically to a known analyte, such as a protein-containing analyte. In one aspect, at least one of the nanoparticles contained in the gel matrix can have a net charge to aid in analyte separation during electrophoresis. In another embodiment, the nanoparticles can each provide a unique SERS-signal that is correlated with binding specificity of the probe of the nanoparticle.

Because a Raman light source can be projected through the gel, the presence of analytes in the sample can be detected without the need to remove the separated analytes from the gel.

In one aspect, the invention gel matrix incorporates composite organic-inorganic nanoparticle (COINs), which comprise a core and a surface, wherein the core comprises a metallic colloid comprising a first metal and a Raman-active organic compound. COINs and methods of making COINs are described in detail herein below.

For use to separate and detect proteins in a sample, such as a biological sample, the invention gel matrix contains SERS-active nanoparticles having a probe that specifically binds to the protein portion of a protein-containing analyte as described herein. Such probes include antibodies, antigens, polynucleotides, oligonucleotides, receptors, ligands, and the like.

Any of the nanoparticles used in an invention gel matrix may further comprise a fluorescent label that contributes to the optical signature of the nanoparticle.

In another embodiment, the invention provides methods for detecting an analyte in a sample comprising contacting a sample containing an analyte with a separation gel, such as an invention gel matrix, under conditions suitable to allow binding to analytes of the probes in one or more SERS-enhancing nanoparticles to form a complexes; separating the complexes from other sample contents by electrophoresis or magnetophoresis; and detecting SERS signals emitted by complexes separated at various locations in the gel (i.e., with or without removal of the complexes from the gel). SERS signals emitted by a particular complex are associated with the presence of a particular analyte. The sample can be a complex biological sample containing a mixture of proteins or protein-containing analytes, in which case the gel matrix will comprise a plurality of different SERS-enhancing nanoparticles to indicate the presence in the sample of different analytes to which the probes in the nanoparticles bind specifically. The sample containing biological targets to be separated is either contacted with the SERS-enhancing nanoparticles prior to introduction of the mixture into the gel for separation, or the sample is introduced into an invention gel already having the SERS-active nanoparticles incorporated therein.

In another embodiment, specificity of the probe in the nanoparticles used in the gel matrix and gel separation methods is unknown and the SERS signal from a particular bound complex provides information regarding the chemical structure of the analyte to which it has bound. Additional information about a bound analyte is obtained by analysis of behavior of the complex in the particular separation medium used (e.g., type of gel, electrical or magnetic conditions of separation), and such information can be compiled with and will supplement the information obtained from analysis of the Raman signals. A compilation of such information for all of the detected analytes can be used to generate a protein profile of the sample.

Separation of the complexes within the gel matrix is accomplished by electrophoresis or magnetophoresis, or a combination of the two, the latter being used especially in the case where the nanoparticle contains a magnetic substance, such as a metal oxide. The complexes can be separated based upon any of the factors usually associated with gel separation techniques, except that separation will depend upon the net differences in charge, weight, and the like, of the analyte/SERS-enhancing nanoparticle complexes, rather than depending upon the differences in the analytes themselves. Consequently, binding of the analyte to one or more of the nanoparticles can be detected by determining a mobility change in the analyte caused by binding of the probe to the analyte. In certain embodiments, at least two of the nanoparticles have different net charges to exert an influence on analyte separation during electrophoresis.

If the analytes of interest are protein-containing analytes, a polyacrylamide gel matrix can be used and the analytes of interest can be selected from antigens, polypeptides, proteins, glycoproteins, lipoproteins, and combinations thereof. The electrophoresis technique used can be either one dimensional or two-dimensional electrophoresis, for example, under non-denaturing conditions. Optionally, the invention gel separation method can further comprise soaking the gel in a chemical enhancer solution, such as a solution of LiCl or NaCl, to further enhance SERS signals from separated analytes. Furthermore, by drying the gel to which a solution of chemical enhancer has been applied, the samples will be concentrated prior to the detecting.

The invention gel separation methods in which SERS signals from the analytes are detected without removing the analytes from the gel are particularly useful for distinguishing between individual analytes when the sample comprises two or more analytes having substantially the same size and/or same charge density. The SERS signals produced by the two or more analytes upon irradiation by laser can be used to distinguish between and provide structural information regarding differences between two or more analytes, even if the two have the same chemical formula. In addition, the SERS signal obtained can distinguish between analytes having substantially the same size and/or same charge density in the sample.

In one aspect, the invention separation methods can further comprise subjecting the analyte to chromatography or isoelectric focusing prior to or following the detection of SERS signals. Information obtained by the SERS analysis can be compared with and/or compiled with the information obtained by the isoelectric focusing and/or the chromatography to further enhance information about a particular protein, or the protein profile of the sample.

In one embodiment of the invention separation methods the SERS-enhancing nanoparticles are COINs, as described herein.

In still another embodiment, the invention provides methods for making an invention gel matrix comprising forming a liquid composition by mixing together a gel-forming liquid, comprising gel-forming particles in a suitable liquid, and a plurality of Raman-enhancing nanoparticles. The Raman-enhancing nanoparticles have a plurality of unique optical signatures and each comprises a probe for binding to an analyte. A solid gel matrix is obtained from the liquid composition using methods known in the art. Although any type of gel-forming particles commonly used to making separation gels can be used in the invention methods, polyacrylamide and agarose gels are commonly used for separating chemical and biological molecules. For use in the invention methods for screening complex biological samples a plurality of SERS-enhancing nanoparticles, each having an attached probe specific for forming a complex with one of a plurality of different analytes, such as the COINs described herein, can be incorporated into the gel.

The principles applied by those of skill in the art in making polyacrylamide and agarose gels, which are the most common stabilizing media used in electrophoresis, include the following. The widespread use of agarose and acrylamide gels stems from the fact that these matrices also act as “molecular sieves” during electrophoresis, restricting the movement of biomolecules according to their size and structure. Agarose and polyacrylamide gels are cross-linked, sponge like structures. Although they are up to 99.5% water, the size of the pores of these gels is similar to that of many proteins and nucleic acids. As molecules are forced through the gel by the applied voltage, larger molecules are retarded by the gel to a greater extent than are smaller molecules. For any particular gel, molecules smaller than a matrix-determined size are not retarded at all; they move almost as if in free solution. At the other extreme, molecules larger than a matrix-determined size cannot enter the gel at all. Therefore, those of skill in the art will tailor gels to sieve molecules of a desired size range by appropriate choice of matrix concentration. The average pore size of a gel is determined by the percentage of solids in the gel and, for polyacrylamide, by the amount of cross-linker as well.

Although there are practical limits to the range of gel densities possible with agarose and ployacrylamide, these two matrices allow the electrophoretic separation of DNA strands anywhere from oligonucleotides only a few base pairs in length to chromosomes or chromosomal fragments as large as several million base pairs long. Polyacrylamide, which makes a small-pore gel, is used to separate polynucleotides from fewer than 5 bases up to approximately 2,000 base pairs in size. Agarose gels, with their large pore size, can be used to separate nucleic acids from 50 to 30,000 base pairs, and, with pulsed-field techniques, up to chromosome- and similar-sized pieces greater than 5×10⁶ base pairs long.

One of the important features of the invention gels and methods of their use is that detection of separated analytes, i.e., separate protein-containing analytes, can be conducted without the necessity to remove the analytes from the gel. Detection can be accomplished by irradiation of the separated analytes in the gel using any of the SERS techniques known in the art. In addition, due to the super sensitivity of SERS analysis, minute amounts of a separated analyte of interest in a biological samples can be detected and determined quantitatively.

In yet another embodiment, the invention provides systems comprising an invention gel matrix that can be used to carry out the invention gel separation and detection methods. Such systems further comprise a sample containing at least one analyte; and an optical detection system suitable for detecting SERS signals from the nanoparticles. The invention systems that use an invention gel matrix may further comprising a computer comprising an algorithm for analysis of the SERS signals obtained from the sample.

In still another embodiment, the invention provides methods for multiplex detection of analytes in a sample by contacting analytes in a sample under conditions suitable to form complexes with a set of probe constructs. Each probe construct comprises a non-nucleic acid probe conjugated with an optically-active polynucleotide barcode containing at least one SERS-active nucleotide, as described herein, to provide a unique optical signature. The unique optical signature can be a unique SERS signature. In addition, each probe construct in the set is specifically designed to have a unique mobility in the chosen electrophoresis medium. The complexes so formed are separated by electrophoresis. After separation, the unique optical signatures are detected, either within a separation gel or after being removed from the separation medium, in a multiplex manner with a suitable detection device. The suitable detection device will depend upon the optical properties of the optically-active barcode. Since each specifically binding probe is conjugated to a known barcode that emits a distinguishable optical signature, such as a SERS signal, individual optical signatures detected from the constructs are thus associated with the identity of a known analyte in the sample.

Since the electrophoretic mobility of a polynucleotide depends at least in part on the number of nucleic acids in the polynucleotide, the unique mobility of individual probe constructs in the set can be achieved by having a varying number of nucleotides in the barcode of individual members of the set. Similarly, variety in the net charge among members of the set can be achieved by selection of the nucleic acids in the polynucleotide barcode. Since the mobility of the complex is determined by the charge density of the overall complex in electrophoresis, many similar analytes can be separated and then detected simultaneously from a single sample when bound to different members of the set of probe constructs due to the unique mobility characteristics of members of the set of probe constructs used.

In addition, the electrophoretic properties of free targets and/or unbound probe constructs will differ from those of bound complexes, it is a simple matter to remove the free targets and/or free unbound probe constructs from the complexes by electrophoresis, for example, prior to detection of bound complexes. In one aspect, the non-nucleic acid probes in the set of probe constructs can be a set of antibodies that bind specifically to known protein-containing targets in a biological sample. In this case, the target analytes will be protein-containing analytes, as described herein. Alternatively, the non-nucleic acid probe in the probe construct may be the true analyte detected, since the binding preferences of a non-nucleic acid probe (e.g., antibody, receptor, and the like) to a protein-containing molecule or complex in a patient sample may be the purpose of the assay.

The separated complexes in this embodiment of the invention can be separated one dimensionally (e.g., chromatography or electrophoresis) or two dimensionally (e.g., first by chromatography or iso-electric focusing and then by electrophoresis). Since the size, surface property and charge density of a target molecule may change after the formation of probed complexes, use of two different separation principles will aid in separation of the complexes from unbound components.

The optical detection procedure or combination of optical detection procedures to be used will depend on the nature of the analytes, the separation device or matrix, as well as the structure and properties of the probe constructs. The separated complexes can be detected by one or a combination of optical techniques selected from adsorption, reflection, polarization, refraction, fluorescence, Raman spectra, SERS, resonance light scattering, grating-coupled surface plasmon resonance, using techniques described herein and as known in the art.

In still another embodiment, the invention provides methods for making a set of active Raman molecular codes that are designed to be synthesized using well-established DNA/peptide chemistry to build molecular complexes. The active Raman molecular codes feature a poly or oligonucleotide backbone, which is itself Raman active, or has Raman tags chemically attached to the backbone at various positions via built-in functional groups to obtain different Raman signatures, without changing chemical compositions. Each molecular complex to be used as a Raman-active molecular code has an active group (e.g., probe) that attaches directly and specifically to a functional group inherent in an amino acid of a biological analyte or to a protein-containing target.

The invention active Raman molecular codes are generated by obtaining a set of molecular backbones, each comprising an organic polymer with two or more chemically reactive moieties at various positions along the backbone; and attaching two or more small molecule Raman-active tags to each backbone in the set at the chemically reactive moieties, wherein the type, number, and relative position of the Raman-active tags along the backbones of members of the set are variously combined to produce a unique Raman signal for each member of the set.

An active group is also conjugated to the backbones in the set, wherein each active group specifically binds to a different type of functional group inherent in a protein-containing analyte. For example, the active group can be a chemical functional group reactive to a chemical moiety inherent in an amino acid in a biological protein-containing molecule. Alternatively, the active group can be a probe, as described herein, that binds specifically to a known protein-containing molecule.

The molecular backbone can comprise any organic polymer that can be synthesized by known chemical techniques. It can be a structure with properties of a biopolymer, such as naturally occurring or synthetic polysaccharides, proteins, amino acids, or a combination thereof. The backbone can also be a Raman-active single stranded or double stranded polynucleotide fragment, either of which is readily synthesized by standard phosphoramidite chemistry. The backbone includes nucleotide analogs that have been chemically modified to accommodate chemical attachment of the Raman-active tags. The locations in the polymeric backbone of the chemically modified nucleotide analogs is varied to vary the location of the Raman-active tags. Examples of that can be introduced into the backbone for this purpose include 2-amino purines. In various aspects, the backbone can comprise 2 to about 1000 nucleotides, about 50 to about 400, or about 10 to about 100 nucleotides. The backbone, depending upon structure and chemical composition, has up to three functions: a support for the Raman-active tags, a source of Raman signal and an enhancer for the Raman-active tags.

Synthesis of the backbones is particularly convenient when the members of the set have a common oligonucleotide backbone except for the location(s) of the chemically modified nucleotide analog(s) used as an attachment point for a Raman-active tag. In this instance, it is still possible to create a set of active molecular Raman codes, each with a unique Raman signature that will be useful to identify thousands of different analytes in a body fluid.

The Raman-active tags incorporated into the invention active molecular Raman codes are small molecules that are highly active in producing a Raman signal and typically have a molecular weight of less than 1 kDa. Raman-active tags that meet these requirements include dyes (e.g., R6G, Tamra, Rox), amino acids (e.g., arginine, methionine, cysteine), nucleic acid bases (e.g., adenine, or guanine), or any combination thereof. Naturally occurring or synthetic compounds having the above-described characteristics, such as suitable molecular weight and Raman characteristics, can also be used. The Raman-active tags can be placed in any position along the molecular backbone and a single backbone can have more than one such tag. Raman signatures of the members of the set can be adjusted by changing the type, number and relative positions of the Raman-active tags along the backbone during synthesis of the molecular Raman codes.

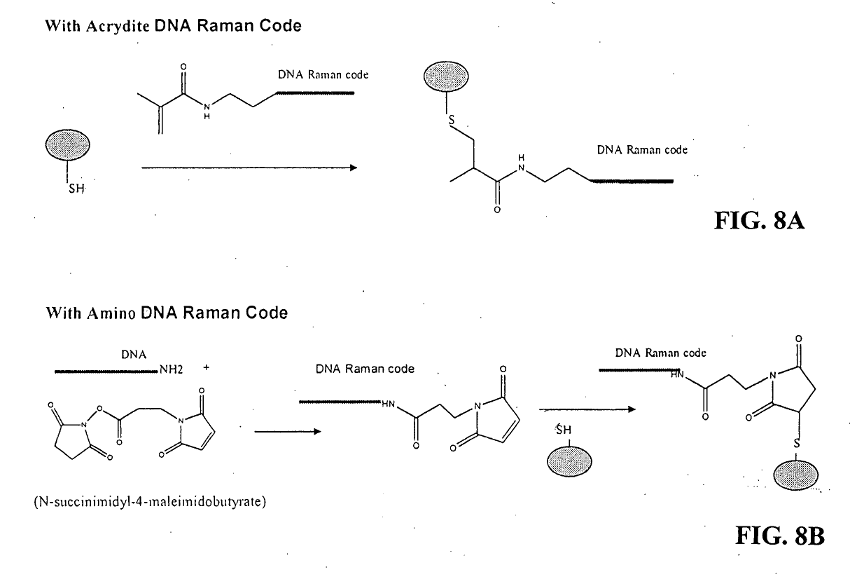

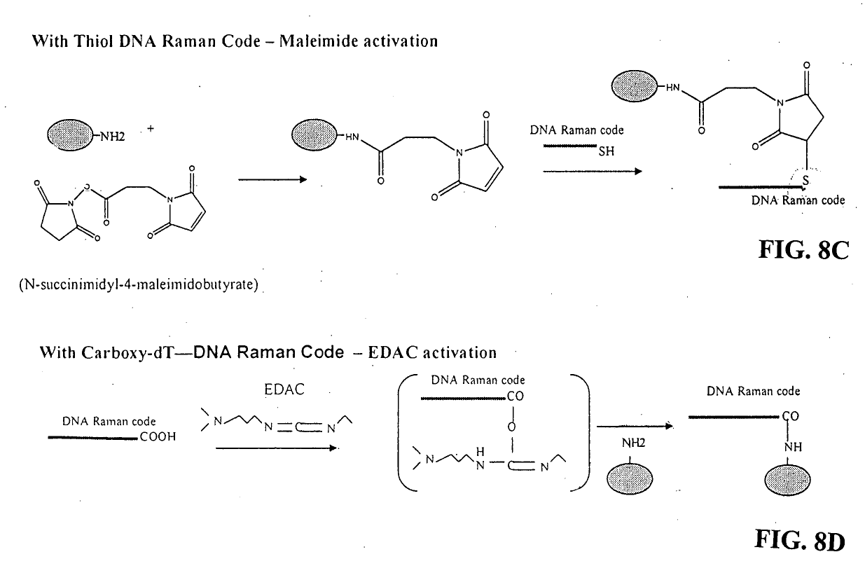

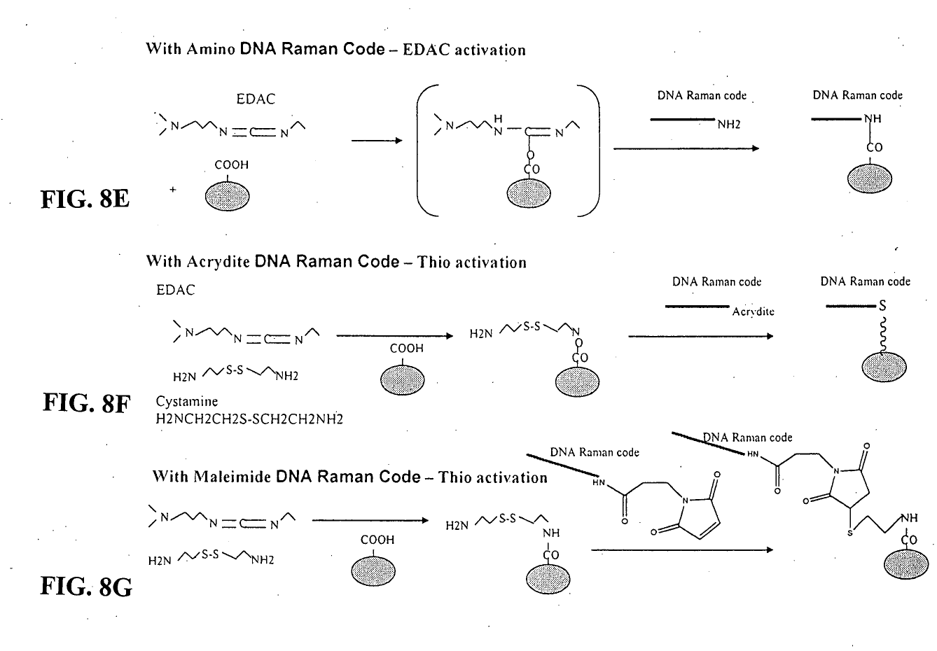

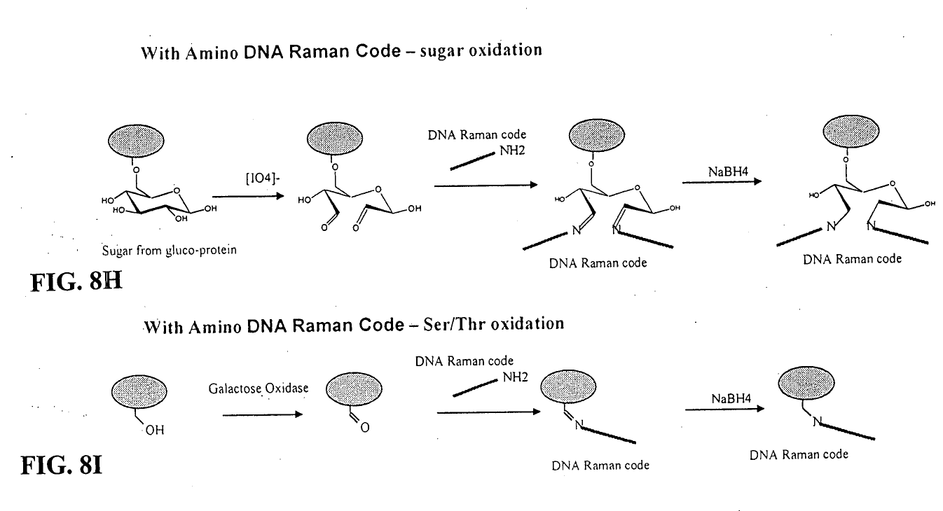

In one aspect, the active group in the invention active molecular Raman codes is a functional group (e.g., acrydite™, amine or thiol group) that is reactive to other functional groups found in specific amino acid residues (e.g., amine, carboxyl, thiol, aldehyde or hydroxyl groups as shown in FIGS. 8A-8I herein. For example, an amino group, when used as the active group, will chemically combine with and identify Lys in an analyte; a sulfhydryl group used as the active group will bind to and identify an amino acid containing a thiol group or Cys; a carboxylic acid active group will bind to and identify an Asp or Glu; and an aldehyde active group will bind to and identify a sugar residue in glycoproteins, using the chemistry shown in FIGS. 8A-8I. Other reagents that can specifically react with protein functional groups can be used in the invention method of generating active molecular Raman codes. In use, when the active group is this type of functional group, a single protein or protein fragment may be the target of and complex with multiple of the active molecular Raman codes, each producing a different Raman signal. In this case, the combination of the molecular Raman codes that bind to a single analyte provides information regarding aspects of the amino acid composition of the analyte.

The second type of active group used in the invention active molecular Raman codes is a probe, such an antibody, receptor, lectin, or a phage-displayed peptide, that binds specifically to a known protein-containing analyte or fragment thereof. Additional examples of probe molecules that can be attached to the polymeric backbones may include but are not limited to oligonucleotides, nucleic acids, antibodies, antibody fragments, binding proteins, receptor proteins, peptides, lectins, substrates, inhibitors, activators, ligands, hormones, cytokines, and the like. This type of active group is conjugated to the backbone using known chemistry, for example DNA/protein chemistry, so that the probe can be used to label or recognize its target molecule (e.g., avoiding steric hindrance to binding).

The Raman active tags used in the invention active molecular Raman codes are selected from a Raman-active dye, amino acid, nucleotide, or a combination thereof. Examples of Raman active amino acids suitable for incorporation into the Raman-active tag include arginine, methionine, cysteine, and combinations thereof. Examples of Raman-active nucleotides suitable for incorporation into the Raman-active tag include adenine, guanine and derivatives thereof.

At least one member of the invention set of active molecular Raman codes may further comprise an enhancer moiety bound to the Raman-active backbone or tag that boosts the intensity of the unique Raman spectrum. For example, a poly(dT) backbone serves as both backbone and enhancer for a dA tag and an amine group attached to a poly(G) Raman tag functions as an enhancer moiety for the tag.

In another embodiment, the invention provides active molecular Raman codes and sets thereof made by the above-described methods. The invention active molecular Raman codes are useful to assay biological samples in several of the methods described herein.

In yet another embodiment, the invention provides methods for assaying a biological sample, as described herein, by use of the invention active molecular Raman codes. To construct a protein profile using the invention active molecular Raman codes, the following exemplary procedure can be followed. Those of skill in the art, using the detailed guidelines provided herein, can devise variations by utilizing different combinations of active groups, molecular backbones and Raman tags. In this embodiment, the protein sample may first, optionally, be digested, for example with trypsin or a variety of sequence-specific proteases known in the art. A combination of different protease digestions and different attachment chemistries can produce a number of sub-samples from the original sample.

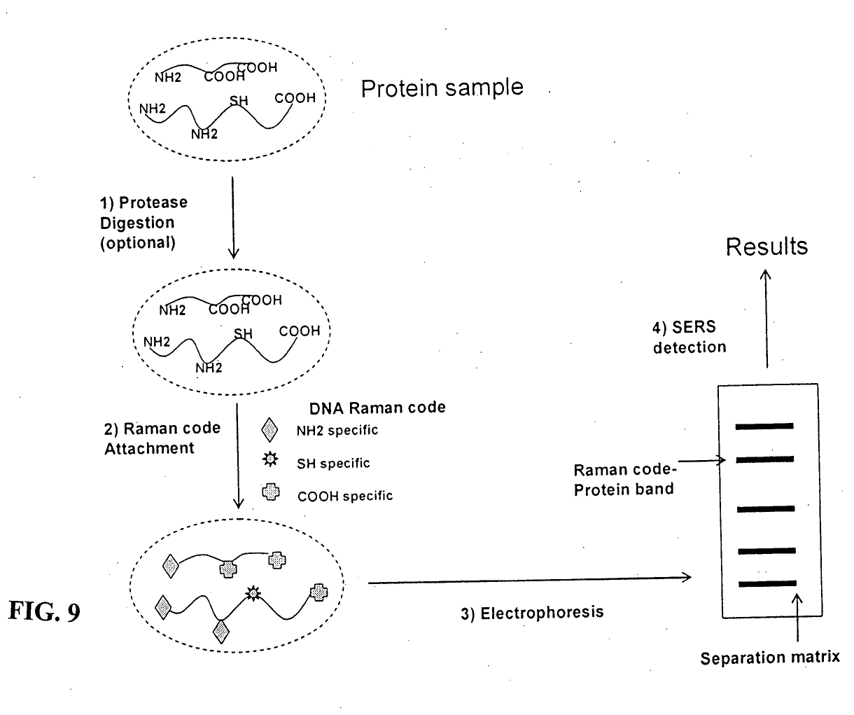

Although any of the invention active molecular Raman code sets can be used, in this exemplary method, a set of three codes is used, each comprising one of the three active groups (i.e., amino, carboxyl and thiol groups) and attached to a different Raman-active backbone, for example a backbone selected from (poly(dA), poly(dG) and polyd(AG) (FIG. 9). The sample (or sub-samples separately) of whole or digested proteins is contacted with the three Raman codes for binding. Then the bound complexes are separated using any suitable separation mechanism, such as electrophoresis (e.g., in a gel matrix), size exclusion chromatography, affinity binding, ion exchange, iso-electric focusing, and the like. When electrophoresis is used, mobility of a complex (in the absence of SDS) depends on the overall size and net negative charges. Capillary electrophoresis is a preferred separation method for detecting small amounts of individual analytes. Each sample or sub-sample is separated in its respective channels or gel matrix lanes. Raman (SERS) signals of separated complexes of Raman code(s)/protein complexes are detected, either in the matrix or transferred out of the matrix before SERS detection. After SERS detection, and correlation of SERS spectra with Raman code information, a great amount of information can be compiled concerning the protein contents of the sample, which is important for protein profiling.

In yet another embodiment, the invention provides methods for determining the presence of an analyte in a sample, wherein cascade binding is used in combination with Raman-active probe constructs for SERS detection. Due to the complexity of biological and chemical systems imperfect (degenerated) reactions or bindings are not uncommon. Studying degenerated binding events can aid identification of useful drugs or disease markers. Currently hundreds of thousands of antibodies against a wide range of agents and biomolecules are available and can be extremely valuable tools for drug screening, disease marker identification, and the like. Accordingly, in this embodiment, the invention method comprises contacting an analyte-containing sample with a first set of probes, such as antibodies or receptors, attached to discrete sites on a solid support to form probe /analyte complexes at the discrete sites. The probe/analyte complexes are then contacted with at least one second set of invention active molecular Raman codes, wherein a subset of the probes of the first set (e.g., antibodies or receptors) is used as the active agents in the second set of probe constructs (i.e., of invention active molecular Raman codes).

The invention method is based on the following assumptions: 1) There is a receptor pool, receptors in the pool are substantially in the same concentration, and each receptor of the pool has a high possibility of binding two or more analytes in a sample when imperfect (degenerated) binding is allowed. 2) There is a sample containing ligands whose abundances are different. For each of the ligands, there are possibly two or more receptors available when degenerated binding is allowed. These assumptions are normally true in a biological system when the receptors are antibodies and the ligands are proteins.

Due to degenerate binding, it is likely that some of the complexes formed by the first binding will be bound by a probe construct (i.e., the analyte is recognized twice by the same antibody or by two different antibodies that bind two epitopes on the analyte or analyte-containing complex). Only those “positives” will be tagged with a Raman-active code. The positively identified analytes can be low abundance analytes in the sample because a protein bound in the first binding event may be relatively enriched (as compared with its concentration in the sample) with respect to the second binding event.

The bound complexes including the Raman-active code are then covered in situ with a thin layer of metal, as described herein, to enhance Raman signals from the Raman-active probe constructs. The metal layer, being in close proximity to the analyte, will produce SERS signals and the complete solid support can be irradiated with a single light source while SERS signals are collected from the bound Raman-code-containing complexes at discrete sites on the solid support, for example by SERS scanning. One or more SERS spectra obtained from a discrete site associates the probe moiety with the presence of a particular analyte in the sample or identifies the probe moiety as having affinity (e.g., heretofore unknown) for a molecule or complex in the sample.

In one aspect, wherein the Raman-active probe constructs comprise oligonucleotide backbones, especially Raman-active oligonucleotide backbones, the method can further comprise amplification of the backbone in bound Raman code-containing complexes on the solid support prior to deposit of the metal layer. For example, PCR™ or terminal transferase reaction amplification can be used to amplify Raman-active backbones, using techniques as known in the art. In the terminal transferase reaction, the dNTP mixture used optionally contain one or more Raman tagged nucleotides or Raman active nucleotides, as described herein.

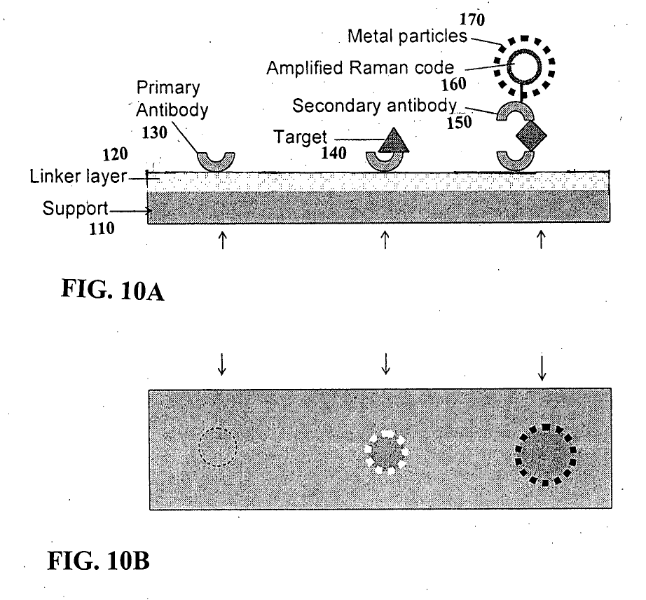

Deposit of a thin layer of metal over the amplified Raman code can be in the form of metal nanoparticles formed in situ to incorporate the amplified Raman codes. Described with reference to FIGS. 10A-B, solid support 110 is coated with a linker layer 120 to attach primary antibody 130. Upon contact with a sample, primary antibody 130 immobilized target 140. Secondary antibody 150 with attached Raman code binds specifically to primary antibody 130 bearing immobilized antigen 140 and the Raman code is amplified using any of the above described techniques. Metal cations are precipitated from a colloidal solution by contact with a reducing agent to form metal nanoparticles 170, which incorporate the amplified Raman code 160.

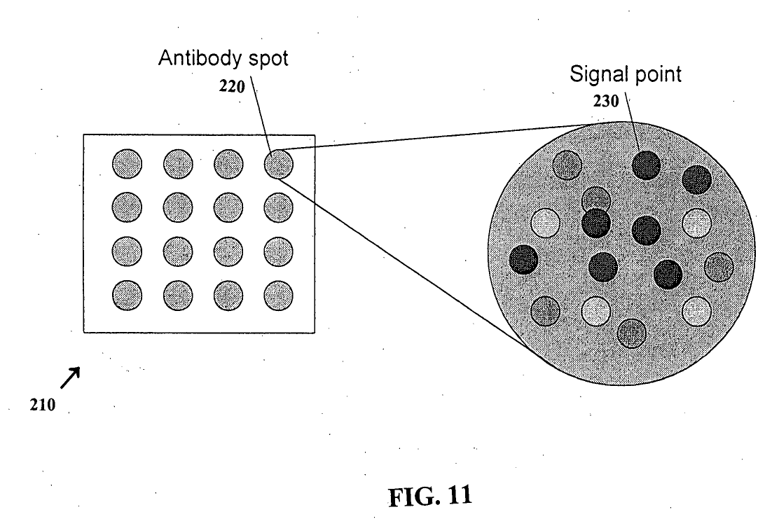

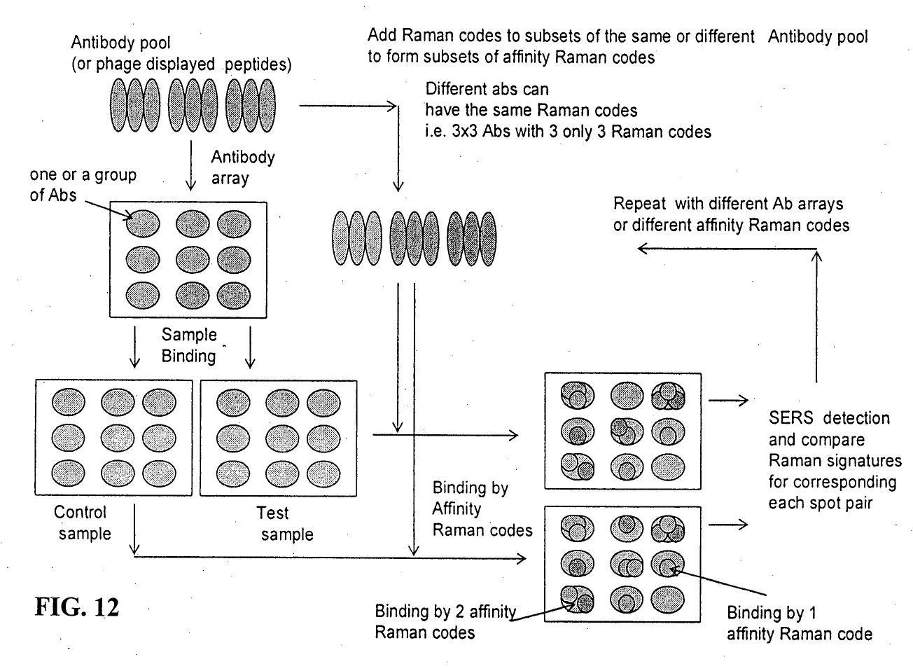

To detect results, SERS signals are counted for individual molecular binding events or signal points emitted from each discrete location on the substrate (e.g. “antibody spot”) As shown in FIG. 11, substrate 210 has a plurality of discrete locations, “i.e., antibody spots”, at which primary antibodies are immobilized on the substrate. A blowup of a single antibody spot illustrates signal point 230, at which a SERS signal is detected due to binding of at least one active molecular Raman code to an analyte immobilized by the primary antibody at the discrete location 220. Multiplex analysis of results obtained by this procedure involves classification of SERS signatures according to Raman code design. Individual signal points can be resolved by performing micro-meter scale SERS scanning and signature analysis as shown by flow chart in FIG. 12.

Antibodies and receptors are non-limiting examples of the probes attached to the discrete locations on the solid support and incorporated into the second set of Raman-active probe constructs. Phage-displayed peptides, nucleic acids, aptamers, ligands, lectins, and combinations thereof can also be used as probes in the invention methods. The sample is not necessarily a body fluid, although it may be, but can comprise any mixed pool of analytes, including proteins, gluco-proteins, lipid proteins, nucleic acids, virus particles, polysaccharides, steroids, and combinations thereof. In one aspect, the sample comprises a pool of body fluid of patients known or suspected of having a particular disease.

For detection of disease markers using the invention method, the method is repeated except that, instead of patient samples representative of a disease, the pool of analytes is made up of corresponding samples (e.g., the same type of body fluid) obtained from normal control patients. The method then further comprises comparing SERS spectra obtained from the patient samples with SERS spectra obtained from the samples of normal control patients to identify a difference, wherein the difference indicates the presence of a disease marker in samples of patients known or suspected of having the disease (FIG. 12).

In one aspect, the first set of probes (e.g., the full set of antibodies) is divided randomly to obtain multiple sub-sets of the probes for use in one of the second sets (e.g., as probe in a active molecular Raman code). Alternatively, the first set of probes can be divided into sub-sets containing equal numbers of probes. In the latter case, each of the probes in a sub-set of the original probe set is used as the active agent in a single second set of active molecular Raman codes and each Raman code in the single sub-set is unique to members of the single sub-set, but each of the second sets contains the same set of Raman codes.

In still another embodiment of the invention methods for assaying a biological sample, the analytes of the sample are separated on a solid support using any of the methods described herein or known in the art, and the separated analytes are contacted with a primary set of active Raman molecular codes so as to allow specific binding of the active Raman molecular codes to one or more protein-containing analytes in the sample to form complexes. Then the complexes so formed are contacted with a secondary Raman code complex so as to amplify Raman signals produced by the active Raman molecular codes in the complexes. Amplified Raman signals produced by the secondary Raman codes are detected and associated with the presence in the sample of the analyte to which the active agent of the active Raman molecular code specifically binds.

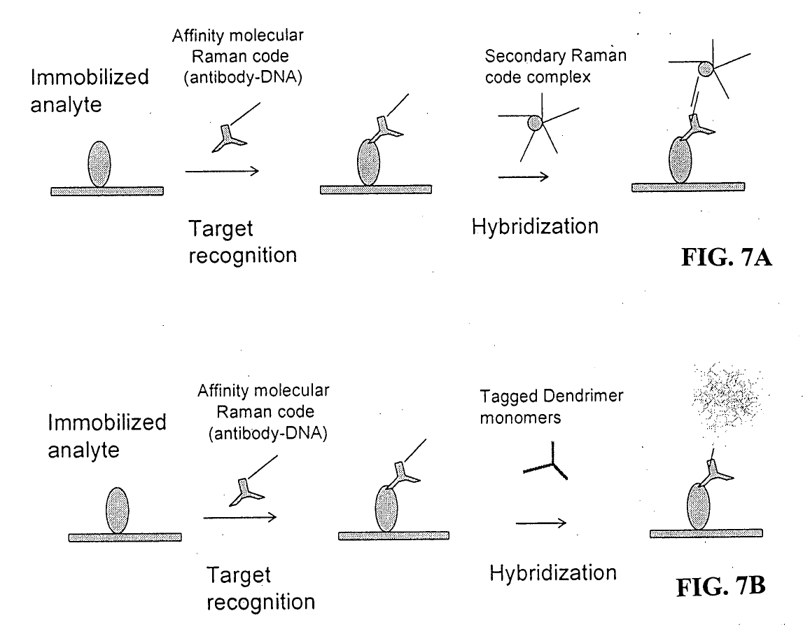

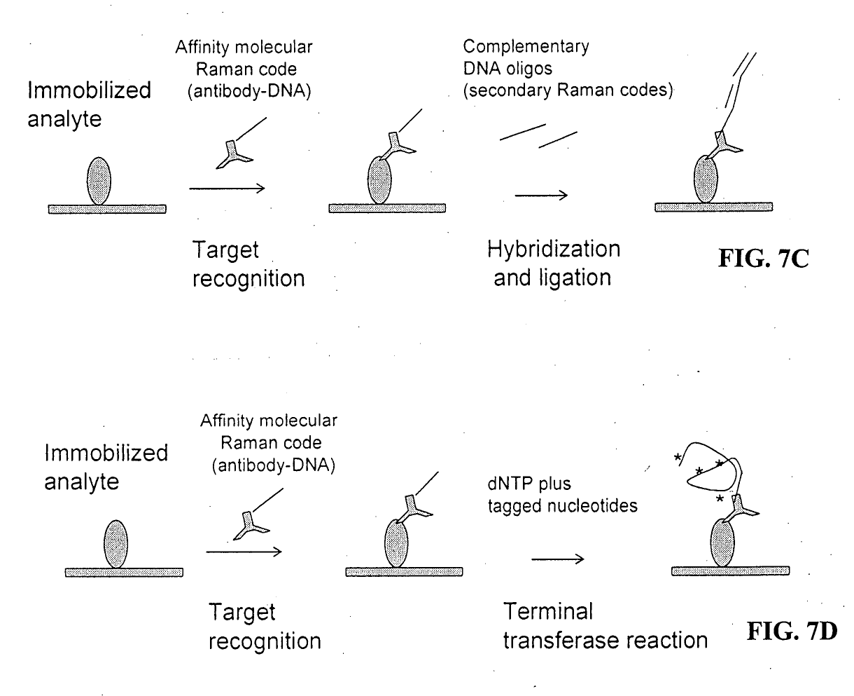

In one aspect, the contacting of the secondary Raman complexes involves chemical association between bound members of the set of active Raman molecular codes and a polynucleotide or oligonucleotide in the secondary Raman codes to amplify the Raman signal. For example, in the case wherein members of the set of active Raman molecular codes comprise an oligonucleotide backbone, optionally Raman-active, and the secondary Raman complexes comprise a complementary oligonucleotide, a selective hybridization reaction between the two is followed by an amplification reaction to amplify the Raman signal. In another aspect, after a selective hybridization, conditions are introduced suitable to cause ligation of the hybridized double-stranded segments to form a linear or branched Raman-active complex that amplifies the Raman signal (FIG. 7C).

In another aspect, wherein the bound members of the set of active Raman molecular codes comprise a Raman-active polynucleotide backbone with a free hydroxyl group at the 3′ end thereof, the method further comprises exposing the bound complexes to dNTPs in the presence of terminal transferase under conditions suitable to form a single stranded Raman-active molecule of hundreds, or thousands, of nucleotides in length to amplify the Raman signal of the backbone. Such a technique is commonly known as “rolling circle amplification.” To further vary the amplified Raman signals, the composition of the dNTPs used to amplify the single stranded DNA backbones of various of the bound complexes can be varied. Raman tagged nucleotides can, optionally, be added to the dNTPs used (FIG. 7D.

In yet another aspect, the secondary Raman complexes can comprise an oligonucleotide or polynucleotides attached to a metal nanoparticle, using methods as described herein (FIG. 7A). The nucleic acid sequence in the secondary Raman sequence is selected to be complementary to at least a portion of the backbone polynucleotide in the active molecular Raman codes. Selective hybridization of the. complementary nucleic acid sequence to the Raman-active molecular backbone will amplify the Raman signal produced upon irradiation. In this case, the amplified Raman signals are SERS signals due to the proximity of the nanoparticles to the analytes. In still another aspect wherein the secondary Raman complexes comprise complementary oligonucleotides or polynucleotides with attached Raman-active tags, the secondary Raman complexes are dendrimers generated from complementary oligonucleotides, as described herein and as known in the art (FIG. 7B). An complementary oligonucleotide in one or more of the dendrimers is selectively hybridized to the polynucleotide backbones in the various active Raman molecular codes to amplify the Raman signal.

It is known that DNA can be amplified by as much as 1×10⁴ fold (rolling circle amplification) to as much as 1×10⁶ fold (PCR™). Thus a combination of DNA amplification and SERS amplification can produce an enhancement factor of 1×10¹⁴ fold. Such an enhancement factor makes possible detection of a single molecule. Typically a protein molecule or a DNA fragment has a dimension of 10-100 nm. If, after amplification, a signal is generated from an area of 1 μm², a chip of 1 cm² would be able to hold 1×10⁸ protein or DNA fragment analytes. Since a laser beam spot can be as small as 1 μm, or less, and concentrations of most cytokines in blood serum are in the range of 1×10⁵ to 1×10¹⁰ molecules/μl (Nature Biotechnology, Apr. 20, 2002:359-356), a minute amount of a sample is sufficient for many qualitative assays as described herein.

In still another embodiment, the invention provides methods for determining the presence of an analyte in a pool of analytes, comprising contacting a pool of analytes with a first set of probes of known binding specificity attached to discrete sites on a solid support to form probe/analyte complexes at the discrete sites. The probe/analyte complexes so formed are then sequentially contacted with multiple second sets of invention active Raman molecular codes, wherein each second set utilizes a sub-set of the probes of the first set as active agents, to form Raman code-containing complexes. The bound Raman code containing complexes are then contacted in situ with metal ions to cover the Raman-code-containing complexes with a thin layer of metal, as described herein, to produce SERS signals upon irradiation of the complexes. SERS signals produced by simultaneously irradiating the bound Raman-code-containing complexes at discrete sites on the solid support are detected and one or more SERS spectra detected is associated with the presence of particular analytes at the discrete site from the sample. Optionally, amplification of the polynucleotide backbone in bound Raman code-containing complexes can be performed, for example by PCR™ or rolling circle amplification as described above, prior to formation of the metal layer to enhance the SERS signals. In one aspect, the SERS signals can be detected using SERS scanning techniques and performing multiplex analysis by classifying SERS spectra according to Raman code designs. In this embodiment, exemplary probes that can be used include antibodies, phage-displayed peptides, receptors, nucleic acids, ligands, lectins, and the like and exemplary analytes that can be detected include proteins, gluco-proteins, lipid proteins, nucleic acids, virus particles, polysaccharides, steroids, and the like. Preferably, the pool of analytes comprises samples of body fluids of patients known or suspected of having a disease. In one aspect, the method is repeated except that the pool of analytes comprises corresponding samples of normal control patients and the method further comprises comparing SERS spectra obtained from the patient pool of analytes with SERS spectra obtained from the pool of analytes of normal control patients to identify a difference, wherein the difference indicates the presence of a disease marker in samples of patients known or suspected of having the disease.

The various invention methods described herein can be used to compile a library of Raman or SERS spectra associated with a particular type of biological sample in healthy individuals as well as in individuals identified as having a particular disease state. Similar libraries can be constructed for a variety of biological samples and a variety of disease states. The Raman or SERS spectra in such a library can then be compared with the results obtained for any individual using invention methods and devices to aid in diagnosing whether the individual has or is likely to have a particular biological phenotype or disease based on their individual spectra (See FIG. 12). Although any biological sample as described herein can be used for such purposes, a particularly suitable biological sample is blood, e.g., blood serum.

The following paragraphs discuss a variety of concepts and terms that will be useful in understanding the various embodiments of the invention.

The term “polynucleotide” is used broadly herein to mean a sequence of deoxyribonucleotides or ribonucleotides that are linked together by a phosphodiester bond. For convenience, the term “oligonucleotide” is used herein to refer to a polynucleotide that is used as a primer or a probe. Generally, an oligonucleotide useful as a probe or primer that selectively hybridizes to a selected nucleotide sequence is at least about 10 nucleotides in length, usually at least about 15 nucleotides in length, for example between about 15 and about 50 nucleotides in length.

A polynucleotide can be RNA or can be DNA, which can be a gene or a portion thereof, a cDNA, a synthetic polydeoxyribonucleic acid sequence, or the like, and can be single stranded or double stranded, as well as a DNA/RNA hybrid. In various embodiments, a polynucleotide, including an oligonucleotide (e.g., a probe or a primer) can contain nucleoside or nucleotide analogs, or a backbone bond other than a phosphodiester bond. In general, the nucleotides comprising a polynucleotide are naturally occurring deoxyribonucleotides, such as adenine, cytosine, guanine or thymine linked to 2′-deoxyribose, or ribonucleotides such as adenine, cytosine, guanine or uracil linked to ribose. However, a polynucleotide or oligonucleotide also can contain nucleotide analogs, including non-naturally occurring synthetic nucleotides or modified naturally occurring nucleotides. Such nucleotide analogs are well known in the art and commercially available, as are polynucleotides containing such nucleotide analogs (Lin et al., Nucl. Acids Res. 22:5220-5234 (1994); Jellinek et al., Biochemistry 34:11363-11372 (1995); Pagratis et al., Nature Biotechnol. 15:68-73 (1997).

The covalent bond linking the nucleotides of a polynucleotide generally is a phosphodiester bond. However, the covalent bond also can be any of numerous other bonds, including a thiodiester bond, a phosphorothioate bond, a peptide-like bond or any other bond known to those in the art as useful for linking nucleotides to produce synthetic polynucleotides (see, for example, Tarn et al., Nucl. Acids Res. 22:977-986 (1994); Ecker and Crooke, BioTechnology 13:351360 (1995)). The incorporation of non-naturally occurring nucleotide analogs or bonds linking the nucleotides or analogs can be particularly useful where the polynucleotide is to be exposed to an environment that can contain a nucleolytic activity, including, for example, a tissue culture medium or upon administration to a living subject, since the modified polynucleotides can be less susceptible to degradation.

As used herein, the term “selective hybridization” or “selectively hybridize,” refers to hybridization under moderately stringent or highly stringent conditions such that a nucleotide sequence preferentially associates with a selected nucleotide sequence over unrelated nucleotide sequences to a large enough extent to be useful in identifying the selected nucleotide sequence. It will be recognized that some amount of non-specific hybridization is unavoidable, but is acceptable provided that hybridization to a target nucleotide sequence is sufficiently selective such that it can be distinguished over the non-specific cross-hybridization, for example, at least about 2-fold more selective, generally at least about 3-fold more selective, usually at least about 5-fold more selective, and particularly at least about 10-fold more selective, as determined, for example, by an amount of labeled oligonucleotide that binds to target nucleic acid molecule as compared to a nucleic acid molecule other than the target molecule, particularly a substantially similar (i.e., homologous) nucleic acid molecule other than the target nucleic acid molecule. Conditions that allow for selective hybridization can be determined empirically, or can be estimated based, for example, on the relative GC:AT content of the hybridizing oligonucleotide and the sequence to which it is to hybridize, the length of the hybridizing oligonucleotide, and the number, if any, of mismatches between the oligonucleotide and sequence to which it is to hybridize (see, for example, Sambrook et al., “Molecular Cloning: A laboratory manual (Cold Spring Harbor Laboratory Press 1989)).

An example of progressively higher stringency conditions is as follows: 2×SSC/0.1% SDS at about room temperature (hybridization conditions); 0.2×SSC/0.1% SDS at about room temperature (low stringency conditions); 0.2×SSC/0.1% SDS at about 42EC (moderate stringency conditions); and 0.1×SSC at about 68EC (high stringency conditions). Washing can be carried out using only one of these conditions, e.g., high stringency conditions, or each of the conditions can be used, e.g., for 10-15 minutes each, in the order listed above, repeating any or all of the steps listed. However, as mentioned above, optimal conditions will vary, depending on the particular hybridization reaction involved, and can be determined empirically.

A “dendrimer” as the term is used herein, are synthetic 3-dimensional molecules that are prepared in a step-wise fashion from simple branched monomer units, the nature and functionality of which can be easily controlled. Formation of dendrimers is described on the world wide web at the address almaden.ibm.com/st/projects/dendrimers.

The disclosed methods and compositions are not limiting as to the type of probe used, and any type of probe moiety known in the art can be attached to barcodes or molecular backbones and used in the disclosed methods. Thus, a “probe moiety” or “probe” as used herein means a molecule or construct that is a specific binding partner for an analyte or type of analyte. Such probes may include, but are not limited to, antibody fragments, affibodies, chimeric antibodies, single-chain antibodies, ligands, binding proteins, receptors, inhibitors, substrates, etc.

In some embodiments, the Raman-active or SERS-active construct used in the invention methods includes an antibody probe. As used herein, the term “antibody” is used in its broadest sense to include polyclonal and monoclonal antibodies, as well as antigen binding fragments of such antibodies. An antibody useful in a method of the invention, or an antigen binding fragment thereof, is characterized, for example, by having specific binding activity for an epitope of an analyte. Alternatively, as explained below, the analyte can be the probe antibody, particularly in embodiments of the invention methods wherein antibodies used as probes (e.g. active agents) are exposed to body fluids to screen a set of antibodies for utility as drug candidates.

The antibody, for example, includes naturally occurring antibodies as well as non-naturally occurring antibodies, including, for example, single chain antibodies, chimeric, bifunctional and humanized antibodies, as well as antigen-binding fragments thereof. Such non-naturally occurring antibodies can be constructed using solid phase peptide synthesis, can be produced recombinantly or can be obtained, for example, by screening combinatorial libraries consisting of variable heavy chains and variable light chains (see Huse et al., Science 246:1275-1281 (1989)). These and other methods of making, for example, chimeric, humanized, CDR-grafted, single chain, and bifunctional antibodies are well known to those skilled in the art (Winter and Harris, Immunol. Today 14:243-246, 1993; Ward et al., Nature 341:544-546, 1989; Harlow and Lane, Antibodies: A laboratory manual (Cold Spring Harbor Laboratory Press, 1988); Hilyard et al., Protein Engineering: A practical approach (IRL Press 1992); Borrabeck, Antibody Engineering, 2d ed. (Oxford University Press 1995)). Monoclonal antibodies suitable for use as probes may also be obtained from a number of commercial sources. Such commercial antibodies are available against a wide variety of targets. Antibody probes can be conjugated to molecular backbones using standard chemistries, as discussed below.

The term “binds specifically” or “specific binding activity,” when used in reference to an antibody means that an interaction of the antibody and a particular epitope has a dissociation constant of at least about 1×10⁻⁶, generally at least about 1×10⁻⁷, usually at least about 1×10⁻⁸, and particularly at least about 1×10⁻⁹ or 1×10⁻¹⁰ or less. As such, Fab, F(ab′)₂, Fd and Fv fragments of an antibody that retain specific binding activity for an epitope of an antigen, are included within the definition of an antibody.

In the context of the invention, the term “ligand” denotes a naturally occurring specific binding partner of a receptor, a synthetic specific-binding partner of a receptor, or an appropriate derivative of the natural or synthetic ligands. The determination and isolation of ligands is well known in the art (Lerner, Trends Neurosci. 17:142-146, 1994). As one of skill in the art will recognize, a molecule (or macromolecular complex) can be both a receptor and a ligand. In general, the binding partner having a smaller molecular weight is referred to as the ligand and the binding partner having a greater molecular weight is referred to as a receptor.

In certain aspects, the invention pertains to methods for detecting an analyte in a sample. By “analyte” is meant any molecule or compound for which a probe can be found. An analyte can be in the solid, liquid, gaseous or vapor phase. By “gaseous or vapor phase analyte” is meant a molecule or compound that is present, for example, in the headspace of a liquid, in ambient air, in a breath sample, in a gas, or as a contaminant in any of the foregoing. It will be recognized that the physical state of the gas or vapor phase can be changed by pressure, temperature as well as by affecting surface tension of a liquid by the presence of or addition of salts etc.

As indicated above, methods of the present invention, in certain aspects, detect binding of an analyte to a Raman-active probe. The analyte can be comprised of a member of a specific binding pair (sbp) and can be a ligand, which is monovalent (monoepitopic) or polyvalent (polyepitopic), usually antigenic or haptenic, and is a single compound or plurality of compounds which share at least one common epitopic or determinant site. The analyte can be a part of a cell such as bacteria or a cell bearing a blood group antigen such as A, B, D, etc., or an HLA antigen or a microorganism, e.g., bacterium, fungus, protozoan, or virus. In certain aspects of the invention, the analyte is charged.

A member of a specific binding pair (“sbp member”) is one of two different molecules, having an area on the surface or in a cavity which specifically binds to and is thereby defined as complementary with a particular spatial and polar organization of the other molecule. The members of the specific binding pair are referred to as ligand and receptor (antiligand) or analyte and probe. Therefore, a probe is a molecule that specifically binds an analyte. These will usually be members of an immunological pair such as antigen-antibody, although other specific binding pairs such as biotin-avidin, hormones-hormone receptors, nucleic acid duplexes, IgG-protein A, polynucleotide pairs such as DNA-DNA, DNA-RNA, and the like are not immunological pairs but are included in the invention and the definition of sbp member.

Specific binding is the specific recognition of one of two different molecules for the other compared to substantially less recognition of other molecules. Generally, the molecules have areas on their surfaces or in cavities giving rise to specific recognition between the two molecules. Exemplary of specific binding are antibody-antigen interactions, enzyme--substrate interactions, polynucleotide interactions, and so forth.

Non-specific binding is non-covalent binding between molecules that is relatively independent of specific surface structures. Non-specific binding may result from several factors including hydrophobic interactions between molecules.

The Raman-active probe constructs described herein as used in the invention methods can be used to detect the presence of a particular target analyte, for example, a nucleic acid, oligonucleotide, protein, enzyme, antibody or antigen. The nanoparticles may also be used to screen bioactive agents, i.e. drug candidates, for binding to a particular target or to detect agents like pollutants. As discussed above, any analyte for which a probe moiety, such as a peptide, protein, oligonucleotide or aptamer, can be designed can be detected in the invention methods by incorporating the probe into the disclosed Raman-active constructs.

The polyvalent ligand analytes will normally be poly(amino acids), i.e., polypeptides and proteins, polysaccharides, nucleic acids, and combinations thereof. Such combinations include components of bacteria, viruses, chromosomes, genes, mitochondria, nuclei, cell membranes and the like.

For the most part, the polyepitopic ligand analytes to which the subject invention can be applied will have a molecular weight of at least about 5,000, more usually at least about 10,000. In the poly(amino acid) category, the poly(amino acids) of interest will generally be from about 5,000 to 5,000,000 molecular weight, more usually from about 20,000 to 1,000,000 molecular weight; among the hormones of interest, the molecular weights will usually range from about 5,000 to 60,000 molecular weight.

The monoepitopic ligand analytes will generally be from about 100 to 2,000 molecular weight, more usually from 125 to 1,000 molecular weight. The analytes include drugs, metabolites, pesticides, pollutants, and the like. Included among drugs of interest are the alkaloids. Among the alkaloids are morphine alkaloids, which includes morphine, codeine, heroin, dextromethorphan, their derivatives and metabolites; cocaine alkaloids, which include cocaine and benzyl ecgonine, their derivatives and metabolites; ergot alkaloids, which include the diethylamide of lysergic acid; steroid alkaloids; iminazoyl alkaloids; quinazoline alkaloids; isoquinoline alkaloids; quinoline alkaloids, which include quinine and quinidine; diterpene alkaloids, their derivatives and metabolites.

The term analyte further includes polynucleotide analytes such as those polynucleotides defined below. These include m-RNA, r-RNA, t-RNA, DNA, DNA-RNA duplexes, etc. The term analyte also includes receptors that are polynucleotide binding agents, such as, for example, restriction enzymes, activators, repressors, nucleases, polymerases, histones, repair enzymes, chemotherapeutic agents, and the like.

The analyte can be a molecule found directly in a sample such as a body fluid from a host. The sample can be examined directly or can be pretreated to render the analyte more readily detectible. Furthermore, the analyte of interest can be determined by detecting an agent probative of the analyte of interest such as a specific binding pair member complementary to the analyte of interest, whose presence will be detected only when the analyte of interest is present in a sample. Thus, the agent probative of the analyte becomes the analyte that is detected in an assay. The body fluid can be, for example, urine, blood, plasma, serum, saliva, semen, stool, sputum, cerebral spinal fluid, tears, mucus, and the like.

As used herein, the term “colloid” refers to metal ions suspended in a liquid, usually water. Typical metals contemplated for use in invention metal colloids and to from nanoparticles include the transparent metals, for example, silver, gold, platinum, aluminum, and the like.

To enhance the Raman spectra produced by Raman-active probes, in certain embodiment of the invention methods, it is contemplated to transform the Raman-active probes to SERS-active probes in situ after binding of the Raman-active probes to the analytes or to a complex containing the analytes. To this purpose, a thin layer of a transparent metal, wherein the layer has a roughened surface, is deposited over the upper layer of the substrate and/or the bound complexes thereon. The roughness features are on the order of tens of nanometers; small, compared to the wavelength of the incident excitation radiation. The small size of the particles allows the excitation of the metal particle's surface plasmon to be localized on the particle. Metal roughness features at the metal surface can be developed in a number of ways; for example; vapor deposition of metal particles or application of metal colloids onto the upper layer of the biosensor. Since the surface electrons of the metal are confined to the particle, whose size is small, the plasmon excitation is also confined to the roughness feature. The resulting electromagnetic field of the plasmon is very intense, greatly enhancing the SERS signal as compared to a Raman signal.

It has been estimated that only 1 in 10 analyte molecules inelastically scatter in Raman Spectroscopy. However, in embodiments of the invention methods wherein the intensity of Raman signal from a scattering molecule is greatly enhanced under SERS conditions, low concentrations of a Raman-active analyte can be detected at concentrations as low as pico- and femto-molar. In some circumstances, the invention methods can be used to detect the presence of a single analyte molecule in a complex biological sample, such as blood serum, by depositing a thin layer of a transparent metal so as to be in contact with the bound complexes containing a Raman label. Gold, silver, copper and aluminum are the transparent metals most useful for this technique.

A roughened metal surface can be produced using one of several methods. The term “a thin metal layer” as used herein means a metal layer deposited by chemical vapor deposition over the bound complexes containing a Raman label. Alternatively, a thin metal layer means a layer of nanoparticles formed by subjecting a colloidal solution of metal cations to reducing conditions to form metal nanoparticles in situ. In some embodiments, the nanoparticles will contain the bound complexes. Alternatively, seed particles, for example attached to the Raman codes, can precipitate formation of the nanoparticles from a metal colloid solution. Metal atoms can also be deposited on the active molecular Raman codes by catalyzed reduction of a metal cation solution using an enzyme tag attached to a probe construct, for example, attached to a molecular backbone or barcode in a probe construct In this context, “thin” means having a thickness of about one-half the wavelength of the irradiating light source (usually a laser) to achieve the benefit of SERS, for example from about 15 nm to about 500 nm, such as about 100 nm to about 200 nm.

In other embodiments, the optical probe constructs or Raman-active probe constructs useful in certain of the invention methods are described as containing “backbones” to which a probe and optically-active tag is attached. In one aspect, Raman code backbones can be formed from polymer chains comprising organic structures, including any combination of nucleic acid, peptide, polysaccharide, and/or chemically derived polymer sequences. In certain embodiments, the backbone can comprise single or double-stranded nucleic acids. In some embodiments, the backbone can be attached to a probe moiety, such as an oligonucleotide, antibody or aptamer. Oligonucleotide mimetics can be incorporated to generate the organic backbone. Both the sugar and the internucleoside linkage, i.e., the backbone, of the nucleotide units can be replaced with novel groups.

In another aspect, molecular probes can be used to hybridize with an appropriate nucleic acid target compound. One example of an oligomeric compound or an oligonucleotide mimetic that has been shown to have excellent hybridization properties is referred to as a peptide nucleic acid (PNA). In PNA compounds, the sugar-backbone of an oligonucleotide is replaced with an amide containing backbone, for example an aminoethylglycine backbone. In this example, the nucleobases are retained and bound directly or indirectly to an aza nitrogen atom of the amide portion of the backbone. Several United States patents that disclose the preparation of PNA compounds include, for example, U.S. Pat. Nos. 5,539,082; 5,714,331; and 5,719,262. In addition, PNA compounds are disclosed in Nielsen et al. (Science, 1991, 254, 1497-15).

In order to distinguish one active molecular Raman code from another, tags can be added directly to the backbone The tags can be read by an imaging modality, for example fluorescent microscopy, FTIR (Fourier transform infra-red) spectroscopy, Raman spectroscopy, electron microscopy, and surface plasmon resonance. Different variants of imaging are known to detect morphological, topographic, chemical and/or electrical properties of tags, including but not limited to conductivity, tunneling current, capacitive current, etc. The imaging modality used will depend on the nature of the tag moieties and the resulting signal produced. Different types of known tags, including but not limited to fluorescent, Raman, nanoparticle, nanotubes, fullerenes and quantum dot tags can be used to identify Raman codes by their topographical, chemical, optical and/or electrical properties. Such properties will vary as a function both of the type of tag moiety used and the relative positions of the tags on the backbone, resulting in distinguishable signals generated for each barcode.

The tags may comprise, for example, Raman-active tags or fluorescent tags, as described herein. Because adjacent tags may interact with each other, for example by fluorescent resonance energy transfer (FRET) or other mechanisms, the signals obtained from the same set of tag moieties may vary depending upon the locations and distances between the tags. Thus, active molecular Raman codes with similar or identical backbones can be distinguishably labeled. In certain embodiments of the invention, the backbone of an active molecular Raman code can be formed of phosphodiester bonds, peptide bonds, and/or glycosidic bonds. For example, standard phosphoramidite chemistry can be used to make backbones comprising DNA chains. Other methods for making phosphodiester linked backbones are known, such as polymerase chain reaction (PCR™) amplification. The ends of the backbone may have different functional groups, for example, biotins, amino groups, aldehyde groups or thiol groups. The functional groups can be used to bind to probe moieties or for attachment of tags. Tags can be further modified to obtain different sizes, electrical or chemical properties to facilitate detection. For example, an antibody could be used to bind to a digoxigenin or a fluorescein tag. Streptavidin could be used to bind to biotin tags.

Where the backbone comprises a peptide moiety, or the tag includes one or more amino acid moieties, the peptide can be phosphorylated for tag modification or for chemical reaction with the tag.

In certain embodiments of the invention, polymeric backbones are generated to which Raman-active tags are chemically attached. The backbone moiety can be comprised of any type of monomer suitable for polymerization, including but not limited to nucleotides, amino acids, monosaccharides or any of a variety of known plastic monomers, such as vinyl, styrene, carbonate, acetate, ethylene, acrylamide, etc. The polymeric backbone can be attached to a probe moiety, such as an oligonucleotide, antibody, lectin or aptamer probe. Where the polymeric backbone is comprised of nucleotide monomers, attachment to an antibody probe would minimize the possibility of binding of both probe and backbone components to different target molecules. Alternatively, in certain embodiments of the invention using nucleotide monomers for the backbone, because a nucleotide-based backbone would itself produce a Raman emission spectrum that could potentially interfere with detection of attached Raman-active tags, a backbone that produces little or no Raman emission signal can be used to optimize signal detection and minimize signal-to-noise ratio.

Current methods for probe labeling and detection exhibit various disadvantages. For example, probes attached to organic fluorescent tags offer high detection sensitivity but have low multiplex detection capability. Fluorescent tags exhibit broad emission peaks, and fluorescent resonant energy transfer (FRET) limits the number of different fluorescent tags that can be attached to a single probe molecule, while self-quenching reduces the quantum yield of the fluorescent signal. Fluorescent tags require multiple excitation sources if a probe contains more than one type of chromophore. They are also unstable due to photo-bleaching. Another type of potential probe tag is the quantum dot. Quantum dot tags are relatively large structures with multiple layers. In addition to being complicated to produce, the coating on quantum dots interferes with fluorescent emission and there are limits on the number of distinguishable signals that can be generated using quantum dot tags. A third type of probe label consists of dye-impregnated beads. These tend to be very large in size, often larger than the size range of the probe molecule. Detection of dye-impregnated beads is qualitative, not quantitative.

By contrast, “Raman-active tags” offer the advantage of producing sharp spectral peaks, allowing a greater number of distinguishable labels to be attached to probes. The use of surface enhanced Raman spectroscopy (SERS) or similar techniques allows a sensitivity of detection comparable to fluorescent tags. In various embodiments of the invention, one or more Raman-active tag moieties is attached to a probe construct (e.g., to a molecular backbone therein) to facilitate detection and/or identification. Non-limiting examples of Raman-active tags of use include TRIT (tetramethyl rhodamine isothiol), NBD (7-nitrobenz-2-oxa-1,3-diazole), Texas Red dye, phthalic acid, terephthalic acid, isophthalic acid, cresyl fast violet, cresyl blue violet, brilliant cresyl blue, para-aminobenzoic acid, erythrosine, biotin, digoxigenin, 5-carboxy-4′,5′-dichloro-2′,7′-dimethoxy fluorescein, TET (6-carboxy-2′,4,7,7′-tetrachlorofluorescein), HEX (6-carboxy-2′,4,4′,5′,7,7′-hexachlorofluorescein), Joe (6-carboxy-4′,5′-dichloro-2′, 7′-dimethoxyfluorescein) 5-carboxy-2′,4′,5′,7′-tetrachlorofluorescein, 5-carboxyfluorescein, 5-carboxy rhodamine, Tamra (tetramethylrhodamine), 6-carboxyrhodamine, Rox (carboxy-X-rhodamine), R6G (Rhodamine 6G), phthalocyanines, azomethines, cyanines (e.g. Cy3, Cy3.5, Cy5), xanthines, succinylfluoresceins, N,N-diethyl-4-(5′-azobenzotriazolyl)-phenylamine and aminoacridine. These and other Raman-active tags can be obtained from commercial sources (e.g., Molecular Probes, Eugene, Oreg.).

In general, it is contemplated that the Raman-active tag may comprise one or more double bonds, for example carbon to nitrogen double bonds. It is also contemplated that the Raman-active tags may comprise a ring structure with side groups attached to the ring structure, such as polycyclic aromatic compounds in general. Compounds with side groups that increase Raman intensity include compounds with conjugated ring structures, such as purines, acridines, Rhodamine dyes and Cyanine dyes. The overall polarity of a polymeric active molecular Raman code is contemplated to be hydrophilic, but hydrophobic side groups can be included. Other tags that can be of use include cyanide, thiol, chlorine, bromine, methyl, phosphorus and sulfur.

In certain embodiments, the Raman-active tags used in the invention methods and constructs can be independently selected from the group consisting of nucleic acids, nucleotides, nucleotide analogs, base analogs, fluorescent dyes, peptides, amino acids, modified amino acids, organic moieties, quantum dots, carbon nanotubes, fullerenes, metal nanoparticles, electron dense particles and crystalline particles, or a combination of any two or more thereof.

Raman-active tags can be attached directly to molecular backbones or other organic moieties used to make invention Raman-active probe constructs, or can be attached via various linker compounds. Nucleotides that are covalently attached to Raman-active tags are available from standard commercial sources (e.g., Roche Molecular Biochemicals, Indianapolis, Ind.; Promega Corp., Madison, Wis.; Ambion, Inc., Austin, Tex.; Amersham Pharmacia Biotech, Piscataway, N.J.). Raman-active tags that contain reactive groups designed to covalently react with other molecules, for example nucleotides or amino acids, are commercially available (e.g., Molecular Probes, Eugene, Oreg.).

In one aspect, The skilled artisan will realize that the Raman-active tags of use are not limited to those disclosed herein, but may include any known Raman-active tag that can be attached to a backbone or probe construct and detected. Many such Raman-active tags are known in the art.

An exemplary method to generate polymeric Raman tags involves anchoring the growing polymeric Raman tag to a solid support, such as porous glass beads, plastics (including but not limited to acrylics, polystyrene, copolymers of styrene and other materials, polypropylene, polyethylene, polybutylene, polyurethanes, TeflonJ, etc.), polysaccharides, nylon, nitrocellulose, composite materials, ceramics, plastic resins, silica, silica-based materials, silicon, modified silicon, carbon, metals, inorganic glasses, optical fiber bundles or any other type of known solid support. One or more linker molecules (such as a carbon atom chain) can be attached to the support. The length of the linker molecule may vary. For example, the linker can be 2-50 atoms in length. Methods for chemical synthesis of polymers are known in the art and may include, for example, phosphoramidite synthesis of oligonucleotides and/or solid-phase synthesis of peptides. Methods of protecting and deprotecting functional groups are also well known in the art, as in the techniques of oligonucleotide or peptide synthesis.

The individual Raman-active tags attached to a single polymeric backbone may each be different. Alternatively, a polymeric Raman label may contain two or more copies of the same Raman-active tag. To maximize the number of distinguishable active molecular Raman codes, it is contemplated that where multiple Raman-active tags are incorporated into a single polymeric backbone they will generally be different, or in different locations on the polymeric backbone. The use of multiple Raman-active tags attached to a single polymeric backbone allows for a very large number of distinguishable active molecular Raman codes to be produced. The average size of a 4-mer Raman label would be about 4000 Daltons. Therefore, polymeric Raman labels would allow probe-target binding with little steric hindrance.

The polymer backbones can be formed from organic structures, for example any combination of nucleic acid, peptide, polysaccharide, and/or chemically derived polymers. The backbone of a polymeric Raman label can be formed by phosphodiester bonds, peptide bonds, and/or glycosidic bonds. For example, standard phosphoramidite chemistry can be used to make backbones comprising DNA chains. Other methods for making phosphodiester-linked backbones are known, such as polymerase chain reaction (PCR™) amplification. The ends of the backbone may have different functional groups, for example, biotins, amino groups, aldehyde groups or thiol groups. These functionalized groups can be used to link two or more sub polymeric units together. Once the polymer backbone is synthesized to the desired length, two or more different Raman-active tags can be introduced sequentially or simultaneously to bind to reactive functional groups contained in the modified residues. Other tags, for example, fluorescent, nanoparticle, nanotube, fullerene or quantum dot tags can be attached to one or more locations along the backbone using methods known in the art and as described herein, to further diversify the Raman signals produced by a set of active molecular Raman codes.

Various methods for cross-linking molecules to nanoparticles are known in the art, and any such known method can be used. For example, by cross linking a carboxyl group with an amine group in the presence of EDAC (1-Ethyl-3-(3-dimethylaminopropyl)carbodiimide), more than one polynucleotide can be attached to a single nanoparticle.

Nucleic acid molecules to be sequenced can be prepared by any standard technique. In one embodiment, the nucleic acids can be naturally occurring DNA or RNA molecules. Where RNA is used, it can be desired to convert the RNA to a complementary cDNA. Virtually any naturally occurring nucleic acid can be prepared and sequenced by the methods of the present invention including, without limit, chromosomal, mitochondrial or chloroplast DNA or messenger, heterogeneous nuclear, ribosomal or transfer RNA. Methods for preparing and isolating various forms of cellular nucleic acids are known (See, e.g., Guide to Molecular Cloning Techniques, eds. Berger and Kimmel, Academic Press, New York, N.Y., 1987; Molecular Cloning: A Laboratory Manual, 2nd Ed., eds. Sambrook, Fritsch and Maniatis, Cold Spring Harbor Press, Cold Spring Harbor, N.Y., 1989). Non-naturally occurring nucleic acids may also be sequenced using the disclosed methods and compositions. For example, nucleic acids prepared by standard amplification techniques, such as polymerase chain reaction (PCR™) amplification, could be sequenced within the scope of the present invention. Methods of nucleic acid amplification are well known in the art.

Nucleic acids can be isolated from a wide variety of sources including, but not limited to, viruses, bacteria, eukaryotes, mammals, and humans, plasmids, M13, lambda phage, P1 artificial chromosomes (PACs), bacterial artificial chromosomes (BACs), yeast artificial chromosomes (YACs) and other cloning vectors.

Proteins or peptides can be made by any technique known to those of skill in the art, including the expression of proteins, polypeptides or peptides through standard molecular biological techniques, the isolation of proteins or peptides from natural sources, or the chemical synthesis of proteins or peptides. The nucleotide and protein, polypeptide and peptide sequences corresponding to various genes have been previously disclosed, and can be found at computerized databases known to those of ordinary skill in the art. One such database is the National Center for Biotechnology Information's Genbank and GenPept databases, which are available on the world wide web. The coding regions for known genes can be amplified and/or expressed using the techniques disclosed herein or as would be know to those of ordinary skill in the art. Alternatively, various commercial preparations of proteins, polypeptides and peptides are known to those of skill in the art.

Another technique for the preparation of polypeptides according to the invention is the use of peptide mimetics for monoclonal antibody production. Mimetics are peptide-containing molecules that mimic elements of protein secondary structure. See, for example, Johnson et al., “Peptide Turn Mimetics” in Biotechnology And Pharmacy, Pezzuto et al., Eds., Chapman and Hall, New York (1993). The underlying rationale behind the use of peptide mimetics is that the peptide backbone of proteins exists chiefly to orient amino acid side chains in such a way as to facilitate molecular interactions, such as those of antibody and antigen. A peptide mimetic is expected to permit molecular interactions similar to the natural molecule. These principles can be used to engineer second generation molecules having many of the natural properties of the targeting peptides disclosed herein, but with altered and even improved characteristics.

Other embodiments of the invention may use fusion proteins. These molecules generally have all or a substantial portion of a targeting peptide, linked at the N- or C-terminus, to all or a portion of a second polypeptide or protein. For example, fusions may employ leader sequences from other species to permit the recombinant expression of a protein in a heterologous host. Another useful fusion includes the addition of an immunologically active domain, such as an antibody epitope, to facilitate purification of the fusion protein. Inclusion of a cleavage site at or near the fusion junction will facilitate removal of the extraneous polypeptide after purification. Other useful fusions include linking of functional domains, such as active sites from enzymes, glycosylation domains, cellular targeting signals or transmembrane regions It is contemplated within the scope of the present invention that virtually any protein or peptide could be incorporated into a fusion protein comprising a probe peptide. Methods of generating fusion proteins are well known to those of skill in the art. Such proteins can be produced, for example, by chemical attachment using bifunctional cross-linking reagents, by de novo synthesis of the complete fusion protein, or by attachment of a DNA sequence encoding the targeting peptide to a DNA sequence encoding the second peptide or protein, followed by expression of the intact fusion protein.

Peptides and polypeptides used in the invention methods and constructs can be synthetically produced. Various automatic synthesizers are commercially available and can be used in accordance with known protocols. See, for example, Stewart and Young, (1984); Tam et al., (1983); Merrifield, (1986); and Barany and Merrifield (1979). Short peptide sequences, usually from about 6 up to about 35 to 50 amino acids, can be readily synthesized by such methods. Alternatively, recombinant DNA technology can be employed wherein a nucleotide sequence which encodes a peptide of the invention is inserted into an expression vector, transformed or transfected into an appropriate host cell, and cultivated under conditions suitable for expression.

The “analytes”, as the term is used herein, includes nucleic acids, proteins, peptides, lipids, carbohydrates, glycolipids, glycoproteins or any other potential target for which a specific probe can be prepared. As discussed above, antibody or aptamer probes can be incorporated into the invention active molecular Raman codes and used to identify any target for which an aptamer or antibody can be prepared. The presence of multiple analytes in a sample can be assayed simultaneously, since each member of a set can be distinguishably labeled and detected. Quantification of the analyte can be performed by standard techniques, well known in spectroscopic analysis. For example, the amount of analyte bound to an invention Raman probe construct can be determined by measuring the signal intensity produced and comparison to a calibration curve prepared from known amounts of similar Raman probe construct standards. Such quantification methods are well within the routine skill in the art.

By “substrate” or “solid support” is meant any material that can be modified to contain discrete individual sites appropriate for the attachment or association of analytes and amenable to at least one detection method. In general, the substrates are selected to allow or enhance the optical detection method contemplated for use thereon, and do not appreciably interfere with signal emissions.

A “substrate” as the term is used herein, includes such well known devices as chips or microtiter plates, may comprise a patterned surface containing individual discrete sites that can be treated as described herein bind to individual analytes or types of analytes. Alternatively, in embodiments wherein the probe Raman construct is attached to the substrate, a correlation between the location of an individual site on the array with the Raman code or probe located at that particular site can be made.

Array compositions may include at least a surface with a plurality of discrete substrate sites. The size of the array will depend on the end use of the array. Arrays containing from about 2 to many millions of different discrete substrate sites can be made. Generally, the array will comprise from two to as many as a billion or more such sites, depending on the size of the surface. Thus, very high density, high density, moderate density, low density or very low density arrays can be made. Some ranges for very high-density arrays are from about 10,000,000 to about 2,000,000,000 sites per array. High-density arrays range from about 100,000 to about 10,000,000 sites. Moderate density arrays range from about 10,000 to about 50,000 sites. Low-density arrays are generally less than 10,000 sites. Very low-density arrays are less than 1,000 sites.

The sites comprise a pattern, i.e. a regular design or configuration, or can be randomly distributed. A regular pattern of sites can be used such that the sites can be addressed in an X-Y coordinate plane. The surface of the substrate can be modified to allow attachment of analytes at individual sites. Thus, the surface of the substrate can be modified such that discrete sites are formed. In one embodiment, the surface of the substrate can be modified to contain wells, i.e. depressions in the surface of the substrate. This can be done using a variety of known techniques, including, but not limited to, photolithography, stamping techniques, molding techniques and microetching techniques. As will be appreciated by those in the art, the technique used will depend on the composition and shape of the substrate. Alternatively, the surface of the substrate can be modified to contain chemically derived sites that can be used to attach analytes or probes to discrete locations on the substrate. The addition of a pattern of chemical functional groups, such as amino groups, carboxy groups, oxo groups and thiol groups can be used to covalently attach molecules containing corresponding reactive functional groups or linker molecules.

Biological “analytes” may comprise naturally occurring proteins or fragments of naturally occurring proteins. Thus, for example, cellular extracts containing proteins, or random or directed digests of proteinaceous cellular extracts, can be used. In this way libraries of procaryotic and eukaryotic proteins can be made for screening the systems described herein. For example libraries of bacterial, fungal, viral, and mammalian proteins can be generated for screening purposes.

The biological analytes can be peptides of from about 5 to about 30 amino acids or about 5 to about 15 amino acids. The peptides can be digests of naturally occurring proteins or random peptides. Since generally random peptides (or random nucleic acids) are chemically synthesized, they may incorporate any nucleotide or amino acid at any position. The synthetic process can be designed to generate randomized proteins or nucleic acids, to allow the formation of all or most of the possible combinations over the length of the sequence, thus forming a library of randomized biological analytes for screening using the invention methods and constructs.

Alternatively, the biological analytes can be nucleic acids. The nucleic acids can be single stranded or double stranded, or a mixture thereof. The nucleic acid can be DNA, genomic DNA, cDNA, RNA or a hybrid, where the nucleic acid contains any combination of deoxyribo- and ribonucleotides, and any combination of bases, including uracil, adenine, thymine, cytosine, guanine, inosine, xanthanine, hypoxanthanine, isocytosine, isoguanine, and base pair analogs such as nitropyrrole and nitroindole, etc.

Methods for oligonucleotide synthesis are well known in the art and any such known method can be used. For example, oligonucleotides can be prepared using commercially available oligonucleotide synthesizers (e.g., Applied Biosystems, Foster City, Calif.). Nucleotide precursors attached to a variety of tags can be commercially obtained (e.g. Molecular Probes, Eugene, Oreg.) and incorporated into oligonucleotides or polynucleotides. Alternatively, nucleotide precursors can be purchased containing various reactive groups, such as biotin, diogoxigenin, sulfhydryl, amino or carboxyl groups. After oligonucleotide synthesis, tags can be attached using standard chemistries. Oligonucleotides of any desired sequence, with or without reactive groups for tag attachment, may also be purchased from a wide variety of sources (e.g., Midland Certified Reagents, Midland, Tex.). Aptamer Probes

“Aptamers” are oligonucleotides derived by an in vitro evolutionary process called SELEX (e.g., Brody and Gold, Molecular Biotechnology 74:5-13, 2000). The SELEX® process involves repetitive cycles of exposing potential aptamers (nucleic acid ligands) to a target, allowing binding to occur, separating bound from free nucleic acid ligands, amplifying the bound ligands and repeating the binding process. After a number of cycles, aptamers exhibiting high affinity and specificity against virtually any type of biological target can be prepared. Because of their small size, relative stability and ease of preparation, aptamers can be well suited for use as probes. Since aptamers are comprised of oligonucleotides, they can easily be incorporated into nucleic acid type backbones. Methods for production of aptamers are well known (e.g., U.S. Pat. Nos. 5,270,163; 5,567,588; 5,670,637; 5,696,249; 5,843,653). Alternatively, a variety of aptamers against specific targets can be obtained from commercial sources (e.g., Somalogic, Boulder, Colo.). Aptamers are relatively small molecules on the order of 7 to 50 kDa.

The term “COINs” as used herein refers to SERS-active nanoparticles incorporated into the invention gel matrix and used in certain other analyte separation techniques described herein. COINs are composite organic-inorganic nanoparticles. These SERS-active probe constructs comprise a core and a surface, wherein the core comprises a metallic colloid comprising a first metal and a Raman-active organic compound. The COINs can further comprise a second metal different from the first metal, wherein the second metal forms a layer overlying the surface of the nanoparticle. The COINS can further comprise an organic layer overlying the metal layer, which organic layer comprises the probe. Suitable probes for attachment to the surface of the SERS-active nanoparticles include, without limitation, antibodies, antigens, polynucleotides, oligonucleotides, receptors, ligands, and the like.

The metal required for achieving a suitable SERS signal is inherent in the COIN, and a wide variety of Raman-active organic compounds can be incorporated into the particle. Indeed, a large number of unique Raman signatures can be created by employing nanoparticles containing Raman-active organic compounds of different structures, mixtures, and ratios. Thus, the methods described herein employing COINs are useful for the simultaneous detection of many analytes in a sample, resulting in rapid qualitative analysis of the contents of “profile” of a body fluid. In addition, since many COINs can be incorporated into a single nanoparticle, the SERS signal from a single COIN particle is strong relative to SERS signals obtained from Raman-active materials that do not contain the nanoparticles described herein as COINs. This situation results in increased sensitivity compared to Raman-techniques that do not utilize COINs.

COINs are readily prepared for use in the invention methods using standard metal colloid chemistry. The preparation of COINs also takes advantage of the ability of metals to adsorb organic compounds. Indeed, since Raman-active organic compounds are adsorbed onto the metal during formation of the metallic colloids, many Raman-active organic compounds can be incorporated into the COIN without requiring special attachment chemistry.

In general, the COINs used in the invention methods are prepared as follows. An aqueous solution is prepared containing suitable metal cations, a reducing agent, and at least one suitable Raman-active organic compound. The components of the solution are then subject to conditions that reduce the metallic cations to form neutral, colloidal metal particles. Since the formation of the metallic colloids occurs in the presence of a suitable Raman-active organic compound, the Raman-active organic compound is readily adsorbed onto the metal during colloid formation. This simple type of COIN is referred to as type I COIN. Type I COINs can typically be isolated by membrane filtration. In addition, COINs of different sizes can be enriched by centrifugation.

In alternative embodiments, the COINs can include a second metal different from the first metal, wherein the second metal forms a layer overlying the surface of the nanoparticle. To prepare this type of SERS-active nanoparticle, type I COINs are placed in an aqueous solution containing suitable second metal cations and a reducing agent. The components of the solution are then subject to conditions that reduce the second metallic cations so as to form a metallic layer overlying the surface of the nanoparticle. In certain embodiments, the second metal layer includes metals, such as, for example, silver, gold, platinum, aluminum, and the like. This type of COIN is referred to as type II COINs. Type II COINs can be isolated and or enriched in the same manner as type I COINs. Typically, type I and type II COINs are substantially spherical and range in size from about 20 nm to 60 nm. The size of the nanoparticle is selected to be about one-half the wavelength of light used to irradiate the COINs during detection.

Typically, organic compounds are attached to a layer of a second metal in type II COINs by covalently attaching organic compounds to the surface of the metal layer Covalent attachment of an organic layer to the metallic layer can be achieved in a variety ways well known to those skilled in the art, such as for example, through thiol-metal bonds. In alternative embodiments, the organic molecules attached to the metal layer can be crosslinked to form a molecular network.

The COIN(s) used in the invention methods can include cores containing magnetic materials, such as, for example, iron oxides, and the like. Magnetic COINs can be handled without centrifugation using commonly available magnetic particle handling systems. Indeed, magnetism can be used as a mechanism for separating biological targets attached to magnetic COIN particles tagged with particular biological probes.

As used herein, “Raman-active organic compound” refers to an organic molecule that produces a unique SERS signature in response to excitation by a laser. A variety of Raman-active organic compounds are contemplated for use as components in COINs. In certain embodiments, Raman-active organic compounds are polycyclic aromatic or heteroaromatic compounds. Typically the Raman-active organic compound has a molecular weight less than about 300 Daltons.

Additional, non-limiting examples of Raman-active organic compounds useful in COINs include TRIT (tetramethyl rhodamine isothiol), NBD (7-nitrobenz-2-oxa-1,3-diazole), Texas Red dye, phthalic acid, terephthalic acid, isophthalic acid, cresyl fast violet, cresyl blue violet, brilliant cresyl blue, para-aminobenzoic acid, erythrosine, biotin, digoxigenin, 5-carboxy-4′,5′-dichloro-2′,7′-dimethoxy fluorescein, 5-carboxy-2′,4′,5′,7′-tetrachlorofluorescein, 5-carboxyfluorescein, 5-carboxy rhodamine, 6-carboxyrhodamine, 6-carboxytetramethyl amino phthalocyanines, azomethines, cyanines, xanthines, succinylfluoresceins, aminoacridine, and the like. These and other Raman-active organic compounds can be obtained from commercial sources (e.g., Molecular Probes, Eugene, Oreg.).

In certain embodiments, the Raman-active compound is adenine, adenine, 4-amino-pyrazolo(3,4-d)pyrimidine, 2-fluoroadenine, N6-benzolyadenine, kinetin, dimethyl-allyl-amino-adenine, zeatin, bromo-adenine, 8-aza-adenine, 8-azaguanine, 6-mercaptopurine, 4-amino-6-mercaptopyrazolo(3,4-d)pyrimidine, 8-mercaptoadenine, or 9-amino-acridine 4-amino-pyrazolo(3,4-d)pyrimidine, or 2-fluoroadenine. In one embodiment, the Raman-active compound is adenine.

When “fluorescent compounds” are incorporated into COINs, the fluorescent compounds can include, but are not limited to, dyes, intrinsically fluorescent proteins, lanthanide phosphors, and the like. Dyes useful for incorporation into COINs include, for example, rhodamine and derivatives, such as Texas Red, ROX (6-carboxy-X-rhodamine), rhodamine-NHS, and TAMRA (5/6-carboxytetramethyl rhodamine NHS); fluorescein and derivatives, such as 5-bromomethyl fluorescein and FAM (5′-carboxyfluorescein NHS), Lucifer Yellow, IAEDANS, 7-Me₂, N-coumarin-4-acetate, 7-OH-4-CH₃ -coumarin-3-acetate, 7-NH₂-4CH₃-coumarin-3-acetate (AMCA), monobromobimane, pyrene trisulfonates, such as Cascade Blue, and monobromotrimethyl-ammoniobimane.

The following paragraphs include further details regarding exemplary applications of Raman-active probe containing constructs (e.g., Raman barcodes, active molecular Raman codes and composite organic-inorganic nanoparticles (COINs)). It will be understood that numerous additional specific examples of applications that utilize such Raman-active probe constructs can be identified using the teachings of the present specification. One of skill in the art will recognize that many interactions between polypeptides and their target molecules can be detected using certain of the disclosed Raman-active probe constructs having a polypeptide as probe. In one group of exemplary applications, such Raman-active constructs that have an antibody as probe moiety are used to detect interaction of the Raman-active antibody labeled constructs with antigens either in solution or on a solid support. It will be understood that such immunoassays can be performed using known methods such as are used, for example, in ELISA assays, Western blotting, or protein arrays, utilizing a Raman-active probe construct having an antibody as the probe and acting as either a primary or a secondary antibody, in place of a primary or secondary antibody labeled with an enzyme or a radioactive compound. In another example, a Raman-active probe construct is attached to an enzyme probe for use in detecting interaction of the enzyme with a substrate.

Another group of exemplary methods uses the Raman-active constructs described herein to detect a target nucleic acid. Such a method is useful, for example, for detection of infectious agents within a clinical sample, detection of an amplification product derived from genomic DNA or RNA or message RNA, or detection of a gene (cDNA) insert within a clone. For certain methods aimed at detection of a target polynucleotide, an oligonucleotide probe is synthesized using methods known in the art. The oligonucleotide is then used to functionalize a Raman-active construct. Detection of the specific Raman label in the Raman-active probe construct identifies the nucleotide sequence of the oligonucleotide probe, which in turn provides information regarding the nucleotide sequence of the target polynucleotide.

In the practice of the present invention, the Raman spectrometer can be part of a detection unit designed to detect and quantify Raman signals of the present invention by Raman spectroscopy. Methods for detection of Raman labeled analytes, for example nucleotides, using Raman spectroscopy are known in the art. (See, e.g., U.S. Pat. Nos. 5,306,403; 6,002,471; and 6,174,677). Variations on surface enhanced Raman spectroscopy (SERS), surface enhanced resonance Raman spectroscopy (SERRS) and coherent anti-Stokes Raman spectroscopy (CARS) have been disclosed.

A non-limiting example of a Raman detection unit is disclosed in U.S. Pat. No. 6,002,471. An excitation beam is generated by either a frequency doubled Nd:YAG laser at 532 nm wavelength or a frequency doubled Ti:sapphire laser at 365 nm wavelength. Pulsed laser beams or continuous laser beams can be used. The excitation beam passes through confocal optics and a microscope objective, and is focused onto the flow path and/or the flow-through cell. The Raman emission light from the Raman-labeled constructs is collected by the microscope objective and the confocal optics and is coupled to a monochromator for spectral dissociation. The confocal optics includes a combination of dichroic filters, barrier filters, confocal pinholes, lenses, and mirrors for reducing the background signal. Standard full field optics can be used as well as confocal optics. The Raman emission signal is detected by a Raman detector, that includes an avalanche photodiode interfaced with a computer for counting and digitization of the signal.

Another example of a Raman detection unit is disclosed in U.S. Pat. No. 5,306,403, including a Spex Model 1403 double-grating spectrophotometer with a gallium-arsenide photomultiplier tube (RCA Model C31034 or Burle Industries Model C3103402) operated in the single-photon counting mode. The excitation source includes a 514.5 nm line argon-ion laser from SpectraPhysics, Model 166, and a 647.1 nm line of a krypton-ion laser (Innova 70, Coherent).

Alternative excitation sources include a nitrogen laser (Laser Science Inc.) at 337 nm and a helium-cadmium laser (Liconox) at 325 nm (U.S. Pat. No. 6,174,677), a light emitting diode, an Nd:YLF laser, and/or various ions lasers and/or dye lasers. The excitation beam can be spectrally purified with a bandpass filter (Corion) and can be focused on the flow path and/or flow-through cell using a 6× objective lens (Newport, Model L6X). The objective lens can be used to both excite the Raman-active probe constructs and to collect the Raman signal, by using a holographic beam splitter (Kaiser Optical Systems, Inc., Model HB 647-26N18) to produce a right-angle geometry for the excitation beam and the emitted Raman signal. A holographic notch filter (Kaiser Optical Systems, Inc.) can be used to reduce Rayleigh scattered radiation. Alternative Raman detectors include an ISA HR-320 spectrograph equipped with a red-enhanced intensified charge-coupled device (RE-ICCD) detection system (Princeton Instruments). Other types of detectors can be used, such as Fourier-transform spectrographs (based on Michaelson interferometers), charged injection devices, photodiode arrays, InGaAs detectors, electron-multiplied CCD, intensified CCD and/or phototransistor arrays.

Any suitable form or configuration of Raman spectroscopy or related techniques known in the art can be used for detection of the Raman-active probe constructs of the present invention, including but not limited to normal Raman scattering, resonance Raman scattering, surface enhanced Raman scattering, surface enhanced resonance Raman scattering, coherent anti-,Stokes Raman spectroscopy (CARS), stimulated Raman scattering, inverse Raman spectroscopy, stimulated gain Raman spectroscopy, hyper-Raman scattering, molecular optical laser examiner (MOLE) or Raman microprobe or Raman microscopy or confocal Raman microspectrometry, three-dimensional or scanning Raman, Raman saturation spectroscopy, time resolved resonance Raman, Raman decoupling spectroscopy or UV-Raman microscopy.

In certain aspects of the invention, a system for detecting the Raman-active probe constructs of the present invention includes an information processing system. An exemplary information processing system may incorporate a computer that includes a bus for communicating information and a processor for processing information. In one embodiment of the invention, the processor is selected from the Pentium® family of processors, including without limitation the Pentium® II family, the Pentium® III family and the Pentium® 4 family of processors available from Intel Corp. (Santa Clara, Calif.). In alternative embodiments of the invention, the processor can be a Celeron®, an Itanium®, or a Pentium Xeon® processor (Intel Corp., Santa Clara, Calif.). In various other embodiments of the invention, the processor can be based on Intel® architecture, such as Intel® IA-32 or Intel® IA-64 architecture. Alternatively, other processors can be used. The information processing and control system may further comprise any peripheral devices known in the art, such as memory, display, keyboard and/or other devices.

In particular examples, the detection unit can be operably coupled to the information processing system. Data from the detection unit can be processed by the processor and data stored in memory. Data on emission profiles for various Raman labels or codes may also be stored in memory. The processor may compare the emission spectra from Raman-active probe constructs in the flow path and/or flow-through cell to identify the Raman-active moiety in the probe construct. The processor may analyze the data from the detection unit to determine, for example, the sequence of a polypeptide bound by a probe of the Raman-active probe constructs of the present invention. The information processing system may also perform standard procedures such as subtraction of background signals or comparison of signals from different samples.

While certain methods of the present invention can be performed under the control of a programmed processor, in alternative embodiments of the invention, the methods can be fully or partially implemented by any programmable or hardcoded logic, such as Field Programmable Gate Arrays (FPGAs), TTL logic, or Application Specific Integrated Circuits (ASICS). Additionally, the disclosed methods can be performed by any combination of programmed general purpose computer components and/or custom hardware components.

Following the data gathering operation, the data will typically be reported to a data analysis operation. To facilitate the analysis operation, the data obtained by the detection unit will typically be analyzed using a digital computer such as that described above. Typically, the computer will be appropriately programmed for receipt and storage of the data from the detection unit as well as for analysis and reporting of the data gathered.

In certain embodiments of the invention, custom designed software packages can be used to analyze the data obtained from the detection unit. In alternative embodiments of the invention, data analysis can be performed using an information processing system and publicly available software packages.

The following examples are intended to illustrate but not limit the invention.

### EXAMPLE 1

To identify cancer-related biomarkers, patient samples and control samples are collected. To increase screening efficiency, multiple patient samples are pooled to normalize the differences. A similar procedure is used for control samples. A pool of 1000 monoclonal antibodies is obtained and is divided into a first set of 200 groups (each with 5 members). Five antibody arrays, each having 200 discrete locations treated to immobilize antibodies, are prepared. The same 1000 antibodies are then grouped in a random order to form a second set of 40 sub-sets (each with 25 members) for use in synthesis of active molecular Raman codes. All 25 members of each of the 40 sub-sets are attached to the same molecular Raman code, using a total of 40 Raman codes to complete synthesis of the active molecular Raman codes. Afterwards, 25 40-member groups of the active molecular Raman codes are formed based on antibodies, each of the 40 members having a different Raman code.

The 25 groups of active molecular Raman codes are then used to detect analytes that have been captured and immobilized at the discrete locations in the first binding. After removal of free Raman codes, Raman codes bound on the array are amplified and SERS scanning is used to collect Raman signatures from all signal points within each discrete location (spot) of an antibody array. The number of signal points with the same signatures are determined. The same procedure is repeated until all 25 Raman code groups have been tested. Finally, differences between patient samples and control samples are analyzed to detect a difference in a patient sample. Such detections yield primary candidates to be cancer markers.

Although the invention has been described with reference to the above example, it will be understood that modifications and variations are encompassed within the spirit and scope of the invention. Accordingly, the invention is limited only by the following claims.

1. A solid gel matrix comprising a solid, separation gel and one or more SERS-enhancing nanoparticles contained in the separation gel the SERS-enhancing nanoparticles having with an attached probe that binds specifically to an analyte.

2. The gel matrix of claim 1 comprising a plurality of the nanoparticles to provide a plurality of unique optical signatures.

3. The gel matrix of claim 2, wherein the SERS-enhancing nanoparticles comprise one or more Raman-active tags independently selected from the group consisting of nucleic acids, nucleotides, nucleotide analogs, base analogs, fluorescent dyes, peptides, amino acids, modified amino acids, organic moieties, quantum dots, carbon nanotubes, fullerenes, metal nanoparticles, electron dense particles and crystalline particles.

4. The gel matrix of claim 1, wherein at least one of the nanoparticles has a net charge.

5. The gel matrix of claim 1, wherein the nanoparticles each provide a unique SERS-signal that is correlated with binding specificity of the probe of the nanoparticle.

6. The gel matrix of claim 1, wherein the Raman-active tag comprises adenine or an analog thereof.

7. The gel matrix of claim 1, wherein the nanoparticles are composite organic-inorganic nanoparticle (COINs) comprising a core and a surface, wherein the core comprises a metallic colloid comprising a first metal and a Raman-active organic compound.

8. The gel matrix of claim 7, wherein the COINs further comprise a second metal different from the first metal forming a layer overlying the surface of the nanoparticle.

9. The gel matrix of claim 8, wherein the COINs further comprise an organic layer overlying the metal layer, which organic layer comprises the probe.

10. The gel matrix of claim 1, wherein the probe is selected from antibodies, antigens, polynucleotides, oligonucleotides, receptors and ligands.

11. The gel matrix of claim 10, wherein the probe comprises a polynucleotide.

12. The gel matrix of claim 1, wherein at least some of the nanoparticles further comprise a fluorescent label that contributes to the optical signature.

13. A method for producing a gel matrix comprising:
a) forming a liquid composition by mixing together
a gel-forming liquid comprising gel-forming particles in a suitable liquid; and
a plurality of Raman-enhancing nanoparticles having a plurality of unique optical signatures, and an attached probe for binding to an analyte; and
b) obtaining a solid gel matrix from the liquid composition.

14. The method of claim 13, wherein the gel matrix comprises a plurality of the SERS-enhancing nanoparticles, each having an attached probe that binds specifically to a known analyte to form a complex.

15. The method of claim 14 wherein the SERS-enhancing nanoparticles are COINs.

16. A method for detecting an analyte in a sample comprising:
contacting a sample containing an analyte with a gel matrix of claim 1 under conditions allowing binding of the probe to the analyte to form a complex;
separating the complex from other sample contents by electrophoresis or magnetophoresis; and
detecting SERS signals emitted by complexes separated at various locations within the gel, wherein a SERS signal emitted by a particular complex is associated with the presence of a particular analyte.

17. The method of claim 16, wherein the gel matrix comprises two or more of the complexes and the signals from the two or more complexes are indicative of the presence of two or more different analytes.

18. The method of claim 16, wherein the SERS signal from a particular complex provides information regarding the chemical structure of the analyte.

19. The method of claim 18, wherein the gel matrix is a polyacrylamide gel and the analytes are selected from antigens, polypeptides, proteins, glycoproteins, lipoproteins, and combinations thereof.

20. The method of claim 16, wherein at least two of the nanoparticles are metal-containing SERS-enhancing nanoparticles having different net charges.

21. The method of claim 20, wherein the SERS-enhancing nanoparticles are COINs.

22. The method of claim 16, wherein the analyte is contained in a biological sample.

23. The method of claim 16, wherein the associating comprises determining a mobility change caused by binding of the probe to the analyte.

24. The method of claim 16, wherein the separating comprises electrophoresis.

25. The method of claim 16, wherein the method further comprising subjecting the analyte to chromatography or isoelectric focusing prior to or following the detecting.

26. The method claim 24, wherein the electrophoresis is one dimensional or two-dimensional electrophoresis under non-denatured conditions.

27. The method of claim 16, wherein the method further comprises soaking the gel in a chemical enhancer solution and drying the gel to concentrate the samples prior to the detecting.

28. The method of claim 16, wherein the sample comprises one or more additional analytes having substantially the same size and/or same charge density and said method comprises associating the optical signals with the identity of the at least one analyte based on altered mobility of the complex in the gel as compared with that of the additional analytes having substantially the same size and/or same charge density in the sample.

29. The method of claim 28, wherein the signals are SERS spectra and the spectra are compared with a SERS database containing SERS spectra of a plurality of analytes to identify bound analytes.

30. The method of claim 29, wherein the SERS spectra of one or more analytes in the sample are compared with a collection of SERS spectra to determine a difference, wherein the difference is associated with a known biological phenotype or disease.

31. The method of claim 16, wherein the sample is a body fluid.

32. The method of claim 31, wherein the sample is blood serum.

33. A system for detecting an analyte in a sample comprising:
a gel matrix of claim 1;
a sample containing at least one analyte; and
an optical detection system suitable for detecting SERS signals from the nanoparticles.

34. The system of claim 33, further comprising a computer comprising an algorithm for analysis of the SERS signals obtained from the sample.

35. A method for multiplex detection of target molecules in a sample, said method comprising:
contacting target molecules in a sample under conditions suitable to allow complex formation of analytes in the sample with a set of probe constructs, each construct comprising a non-nucleic acid probe conjugated with an optically-active nucleic acid barcode comprising at least one SERS-active nucleotide and having both a unique mobility in electrophoresis and a unique optical signature;
separating the complexes by electrophoresis;
detecting the unique optical signatures in a multiplex manner with a suitable detection device; and
associating individual optical signatures from the constructs with the identity of the corresponding analytes in the sample.

36. The method of claim 35, wherein the unique mobility results from the constructs in the set having varying number of nucleotides in the barcode.

37. The method of claim 35, wherein at least some of the constructs have a net charge.

38. The method of claim 35, further comprising separating free targets and/or free unbound probe constructs from the complexes by electrophoresis.

39. The method of claim 35, wherein the non-nucleic acid probes are antibodies that bind specifically to known protein-containing targets.

40. The method of claim 35, wherein the separated complexes are detected by optical techniques selected from adsorption, reflection, polarization, refraction, fluorescence, Raman spectra, SERS, resonance light scattering, grating-coupled surface plasmon resonance and combinations thereof.

41-93. (canceled)
